(19) United States
(12) Patent Application Publication
(10) Pub. No.: US 2019/0343860 A1
(43) Pub. Date: Nov. 14, 2019

(54) HIGHLY SELECTIVE ADENOSINE A3 RECEPTOR SUBTYPE AGONISTS FOR THE PREVENTION AND TREATMENT OF NEURODEGENERATIVE DISORDERS

(71) Applicant: Saint Louis University, St. Louis, MO (US)

(72) Inventor: Daniela Salvemini, Chesterfield, MO (US)

(73) Assignee: Saint Louis University, St. Louis, MO (US)

(21) Appl. No.: 16/096,888

(22) PCT Filed: Apr. 25, 2017
(86) PCT No.: PCT/US2017/029297

§ 371 Date: Oct. 26, 2018

Related U.S. Application Data
(60) Provisional application No. 62/327,543, filed on Apr. 26, 2016.

(51) Int. Cl. A61K 31/7076; A61P 25/02; A61P 25/28

(52) U.S. Cl. CPC A61K 31/7076; A61P 25/28; A61P 25/02

(57) ABSTRACT

The disclosure provides methods and compositions for inhibiting neurodegeneration by administering an A₃AR agonist that ameliorates mitochondrial injury and dysfunction

This application claims benefit of priority to U.S. Provisional Application Ser. No. 62/327,543, filed Apr. 26, 2016, the entire contents of which are hereby incorporated by reference.

### BACKGROUND

### 1. Field

This disclosure relates to the fields of medicine and cell biology. More specifically, the disclosure is directed to the use of drugs that are a highly-selective agonist for the adenosine A3 receptor (A₃AR) subtype in the prevention and treatment of neurodegeneration in a variety of disease states.

### 2. Related Art

It is known that a variety of neurodegenerative conditions are caused, at least in part, by dysfunction of neuronal mitochondria that results in an energy deficit. No practical drug therapy is known for the prevention or treatment of neurodegenerative conditions.

It is also known that nerve cells and other cell types express receptors on their membranes that have adenosine as their natural ligand. There are known to be four adenosine receptor subtypes (A₁AR, A_{2A}AR, A_{2B}AR, and A₃AR). Drug-like molecules are known that have relatively high selectivity for binding to each of the four subtypes. In particular, highly-selective agonists for the A₃AR are known to have diverse pharmacological actions.

### SUMMARY

Thus, in accordance with the present disclosure, there is provided a method of treating or preventing chemotherapy-induced peripheral neuropathy (CIPN) in a subject comprising administering to said subject an A₃AR agaonist. The CIPN may be due to anti-cancer chemotherapy, such as a taxane chemotherapeutic (e.g., paclitaxel), a platinum-complex chemotherapeutic (e.g., oxaliplatin), a vinca alkaloid chemotherapeutic (e.g., vincristine), or a proteasome inhibitor chemotherapeutic (e.g., bortezomib). The CIPN may be due to anti-viral chemotherapy, such as anti-HIV chemotherapy. The A₃AR agonist may be IB-MECA or Cl-IB-MECA, or an adenosine methanocarba derivative including but not limited to, MRS5698, MRS5980, or MRS7154. The subject may be a human, or a non-human mammal.

Also provides is a method of treating or preventing diabetic peripheral neuropathy in a subject comprising administering to said subject an A₃AR agonist. Another embodiment involves a method of treating or preventing neurodegeneration in a subject comprising administering to said subject an A3AR agaonist, such as neurodegeneration due to Alzheimer's disease, Parkinson's disease, Huntington's disease, amyotrophic lateral sclerosis, or Leber's optic neuropathy. The A3AR agonist may be IB-MECA or Cl-IB-MECA, or an adenosine methanocarba derivative including but not limited to MRS5698, MRS5980, or MRS7154. The subject may be a human, or a non-human mammal.

An additional embodiment involves a method preventing or treating oxaliplatin-induced ototoxicity (e.g., deafness, tinnitus, hyperacusia) in a subject comprising administering to said subject an A₃AR agonist. Further, there is disclosed a method of treating or preventing spinocerebellar degeneration in a subject comprising administering to said subject an A₃AR agaonist. The A₃AR agonist may be IB-MECA or Cl-IB-MECA, or an adenosine methanocarba derivative including but not limited to MRS5698, MRS5980, or MRS7154. The subject may be a human, or a non-human mammal.

With respect to chemotherapy embodiments, the chemotherapeutic and said A₃AR agonist are delivered at the same time. The chemotherapeutic or drug and the A₃AR agonist may or may not be co-formulated. If not co-formulated, the chemotherapeutic or drug and the A₃AR agonist may be delivered at distinct times, such as where the chemotherapeutic or drug is delivered before said A₃AR agonist, or where chemotherapeutic or drug is delivered after said A₃AR agonist. The chemotherapeutic or drug and the A₃AR agonist may be delivered in alternating administrations. The chemotherapeutic or drug may be delivered over a period of one week, two weeks, three weeks, four weeks, one month, two months, three months, four months, five months, six months, seven months, eight months, nine months, ten months, eleven months, one year, two years or three years.

The A₃AR agonist in any of the preceding embodiments may be delivered over a period of one week, two weeks, three weeks, four weeks, one month, two months, three months, four months, five months, six months, seven months, eight months, nine months, ten months, eleven months, one year, two years or three years. The A₃AR agonist may be delivered by continuous infusion, such as by an implanted pump.

Any of the preceding methods may be used in combination with an additional “traditional” therapy that prevents or treats CIPN, neurodegeneration or neuropathy.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed.

FIG. 1. Graph showing the incidence (number per centimeter of epidermal border) of intraepidermal nerve fibers (IENFs) in normal control rats (“Naive”), rats treated with paclitaxel or oxaliplatin alone (open bars), and rats treated with paclitaxel+MRS5698 or oxaliplatin+MRS5698 (black bars), a highly-selective A₃AR agonist. Rats treated with paclitaxel or oxaliplatin alone had statistically significantly fewer IENFs than normal Naive rats. Rats treated with pachtaxel+MRS5698 or oxaliplatin+MRS5698 had significantly more IENFs than the rats treated with paclitaxel or oxaliplatin alone, thus demonstrating a neuroprotective effect. Means±SEM. n=10 adult male Spraque-Dawley rats/group, *p<0.05 vs. Naive group, ! p<0.05 vs. paclitaxel alone or oxaliplatin alone; Bonferroni-corrected t-tests.

FIGS. 2A-B. A₃AR agonists attenuate CIPN by protecting mitochondrial function in PNSAs. Oxaliplatin administration for 5 days (10 mg/kg cumulative dose) produces mechano-hypersensitivity (reductions in Paw Withdrawal Threshold (g); FIG. 2A) that is associated with mitochondrial dysfunction (reduced ATP production) in the peripheral sensory afferents (PNSAs; FIG. 2B). Administration of the A₃AR agonist, MRS5698 (0.1 mg/kg/d, i.p.) concomitant with oxaliplatin prevents the deficiencies in mitochondrial ATP production (FIG. 2B) & the development of mechano-hypersensitivity (FIG. 2A). Mean±SEM, n=6/group, ANOVA with Bonferroni comparisons. *P<0.05 vs. day 0; #P<0.05 vs. vehicle &†P<0.05 vs. Oxaliplatin. (FIG. 2A) Janes et al, BBI, 2015³; (FIG. 2B) Janes et al, Pain 2016, in preparation.

FIGS. 3A-C. Raman Imaging of “Simple” Multi-component Samples. Raman scattering occurs when monochromatic light interacts with vibrating bonds in molecules. All organic molecules are Raman active and every molecule has a unique Raman spectrum. (FIG. 3A) Raman Imaging of Pharmaceutical Tablet: Aspirin (gray), Paracetamol (dark gray), Caffeine (very dark gray), Cellulose (very light gray), Tablet coating (light gray). (FIG. 3B) A full Raman spectrum is measured at each point on the sample. The Raman spectra indicate the chemical composition at each spot on the sample and can be deconvoluted to visualize the five different chemical components. (FIG. 3C) For well-defined chemical systems this is very straightforward, since each chemical component is spectroscopically and spatially resolved.

FIGS. 4A-B. Raman Imaging of Cells and Tissues: (FIG. 4A) All biomolecules are Raman active, so Raman spectra of cells are much more complex, but a lot of chemical information can still be mined based on spectral features that are diagnostic of chemical classes. (FIG. 4B) Confocal Raman imaging of cells interrogates the chemical composition at each site and image analysis can be used to define regions of the cells that have similar chemical composition. This approach can be used to locate organelles and other biomolecules without the need for labeling.

FIGS. 5A-B. Imaging in the Cellular “Raman-Silent Region.” (FIG. 5A) Despite the high chemical complexity of a cell, no natural biomolecules have peaks in the 2000-2500 cm⁻¹ region of the spectrum. This is often referred to as the “Raman Silent Region.” (FIG. 5B) Many synthetic organic molecules contain triple bonds which give rise to peaks in the “Raman Silent Region.”

FIGS. 6A-C. Intracellular accumulation of MRS5698. (FIG. 6A) The triple bond in MRS5698 gives a peak in the silent region around 2227 cm⁻¹. This was used to track their location in CHO cells (FIGS. 6B-C). The C—H stretching (light gray) indicates the C—H bond in all biomolecules within the cell (FIG. 6B). As can be seen when CHO cells are treated with MRS5698 (0.2 μM) and the spectra is filtered to the −2220 cm⁻¹ region following, MRS5698 appears to be enriched in a subset of the intracellular space (FIG. 6C). These results are from at least two independent Raman imaging experiments

FIGS. 7A-C. Intracellular accumulation of MRS5980. (FIG. 7A) The triple bond in MRS5980 gives a peak in the silent region around 2224 cm⁻¹. This was used to track their location in CHO cells (FIGS. 7B-C). The C—H stretching (light gray) indicates the C—H bond in all biomolecules within the cell (FIG. 7B). As can be seen when CHO cells are treated with MRS5980 (1 μM) and the spectra is filtered to the −2220 cm⁻¹ region following, MRS5980 appears to be enriched in a subset of the intracellular space (FIG. 7C). These results are from at least two independent Raman imaging experiments.

FIGS. 8A-B. MRS5698 accumulates at mitochondria In CHO cells (FIG. 8A) & mouse BV2 microglia (FIG. 8B), MRS5698 (0.25 μM) localizes in intracellular regions corresponding to the mitochondrial cytochrome c signal (750 cm⁻¹).

FIG. 9. A₃AR is present in mitochondrial fractions of various rat tissues. Representative images of 2-3 Western blots of subcelluar mitochondrial fractions enriched by-differential centrifugation & Optiprep gradients.

FIGS. 10A-E. A₃AR in mitochondria of astrocytes & microglia. STED microscopy reveals the presence of A3AR (light gray) within the TOMM20-labeled (dark gray) outer mitochondrial membrane of rat cortical astrocytes (FIGS. 10A-B) & mouse microglia (FIGS. 10C-D). (FIG. 10E) Three-dimensional rendering of the z-stack images of a BV2 mitochondria further demonstrates A₃AR is embedded within the outer mitochondrial membrane. After STED, maximum resolutions of 52 nm & 107 nm were achieved for Oregon Green 488 (A₃AR) & Cy3 (TOMM20), respectively.

FIGS. 11A-D. A₃AR in isolated rat spinal mitochondria & intact rat peripheral nerve mitochondria. TEM images show that immunogold-labeled A₃AR (black dots) is expressed in the outer membrane of mitochondria isolated from rat spinal cord (FIGS. 11A-B). This is in contrast to the distribution of the inner mitochondrial membrane protein, COXIV (FIG. 11C). A₃AR signal is similarly localized to the outer mitochondrial membrane of intact rat saphenous nerves (FIG. 11D). Images are representative of 3-8 images.

FIG. 12. MRS5980 reduces ADP-dependent dissipation of mitochondrial membrane potential (ΔΨm). ADP (1 mM) in the presence of Complex I and II substrates reduced the ΔΨm in isolated mouse liver mitochondrial (decreased TMRM signal). The degree of dissipation ΔΨm was lessened with MRS5980 (10 μM) treatment prior to adding ADP n=1, 10,000 mitochondria counted/sample.

FIG. 13. MRS5980 reduces calcium-dependent dissipation of mitochondrial ΔΨm. The addition of Ca²⁺ (0.5-15 μM) to isolated mouse liver mitochondria reduced the mitochondria ΔΨm in (decreased TMRM signal). The mitochondrial ΔΨιη was sustained with MRS5980 (10 μM) treatment. n=1, 10,000 mitochondria counted/sample.

FIG. 14. Direct application of MRS5980 to PNSA mitochondria reverses oxaliplatin-induced deficiencies in ATP production. Saphenous nerves explants were harvested on day 25 from rats treated with oxaliplatin or its vehicle. In explants from vehicle-treated rats, direct application of MRS5980 (1 μM) modestly enhanced ATP production (Veh—MRS5980) following stimulation of Complex I and II compared to explants treated with the vehicle of MRS5980 (Veh-Veh). Impressively, in explants from oxaliplatin-treated rats, direct MRS5890 treatment mitigated the loss of ATP production in mitochondria induced by the oxaliplatin. Mean±SEM, n=6/group *P=0.019 vs, t-test

### DESCRIPTION OF ILLUSTRATIVE EMBODIMENTS

Anti-cancer chemotherapeutic drugs in the taxane, vinca alkaloid, platinum-complex, and proteasome inhibitor classes, among others, have as their chief dose-limiting side effect a distal, symmetrical peripheral neuropathy (called CIPN) that is often accompanied by neuropathic pain. Similar neuropathies are found in patients treated with false nucleoside anti-HIV chemotherapeutics and in patients with diabetes.

It is known that all of these peripheral neuropathies are accompanied by degeneration of primary afferent sensory neuron axons and that this degeneration begins at the distal most portion of the axon, which for those sensory axons that innervate the skin is known as the intraepidermal nerve fiber (IENF). Experiments in animal models of these conditions have shown that IENF degeneration is accompanied by dysfunction of the neuronal mitochondria (Bennett et al, 2014; Zenker et al, 2013).

Animal models of diseases that are associated with the degeneration of nerve cells in the central nervous system (including but not limited to Parkinson's disease, Alzheimer's disease, Huntington's disease, spinocerebellar degeneration, and amyotrophic lateral sclerosis) have also provided evidence that mitochondrial dysfunction is a key factor (Carvalho et al, 2015; Cozzolino et al, 2015; Matilla-Duenas et al., 2014).

Moreover, it is hypothesized that mitochondrial dysfunction is the cause of degeneration of the ear's hair cells in patients with deafness, tinnitus and hyperacusis after treatment with platinum-complex anti-cancer chemotherapeutics and certain antibiotics (Devarajan et al, 2002; Guan, 2011).

Animal research has shown that drugs that are highly-selective A3AR agonists can prevent and treat CIPN and diabetic peripheral neuropathy. The effect manifests as prevention or inhibition of the chemotherapy-induced decrease in mitochondrial adenosine triphosphate (ATP) production.

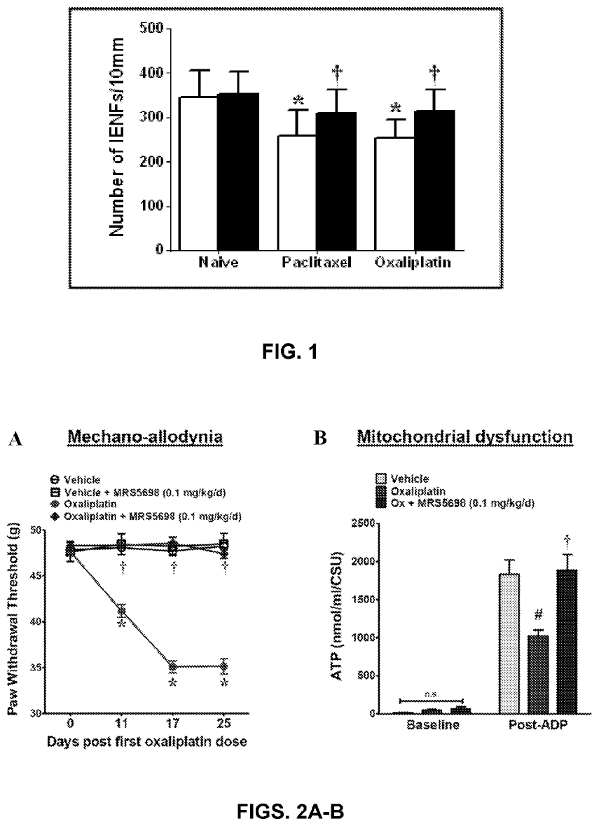

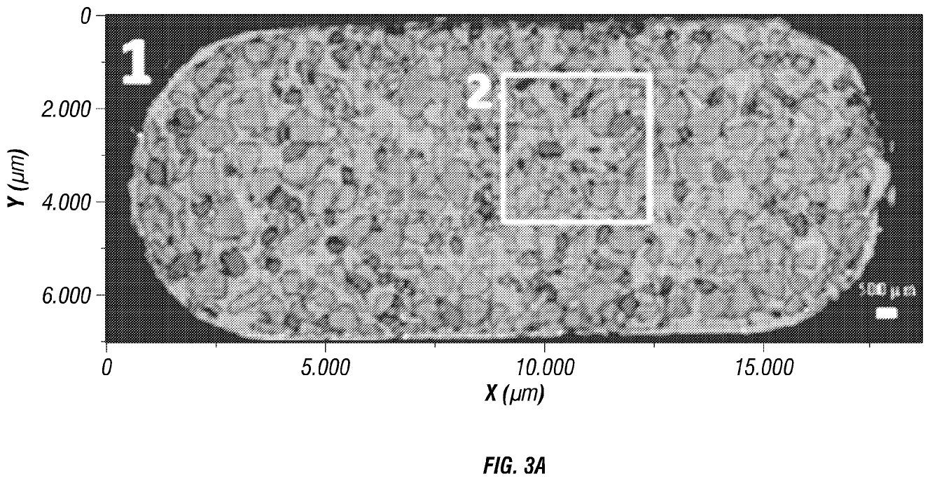

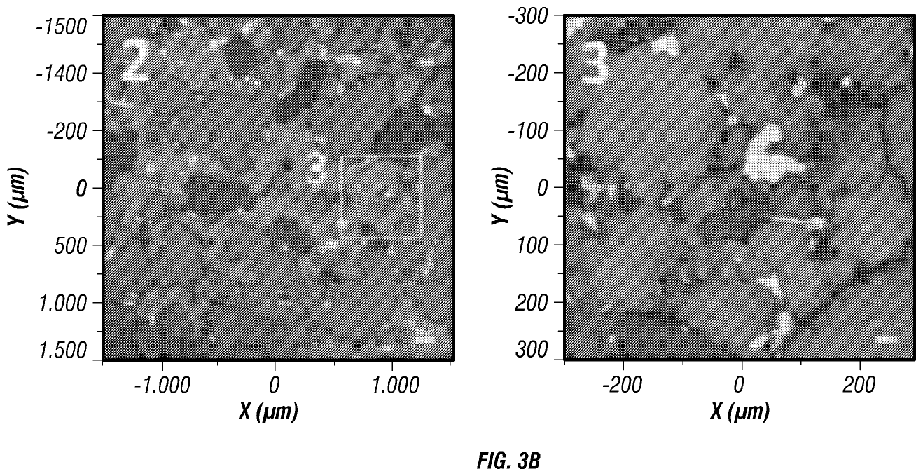

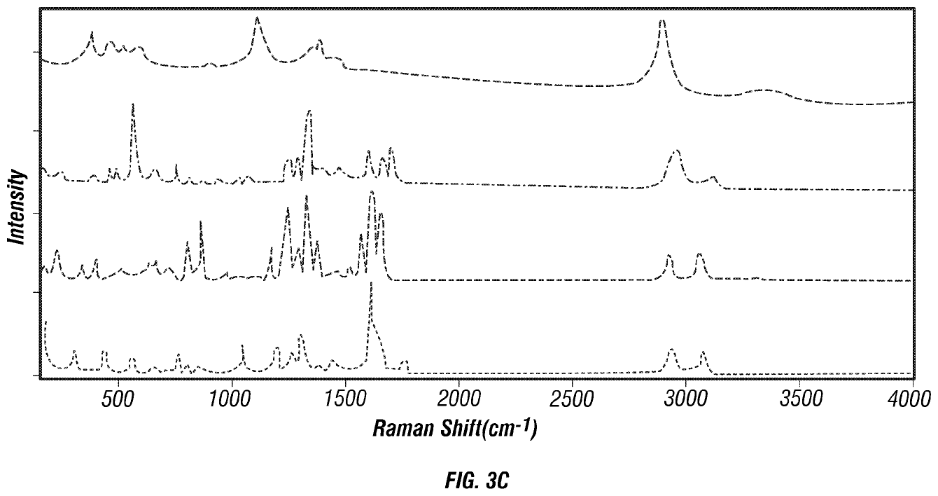

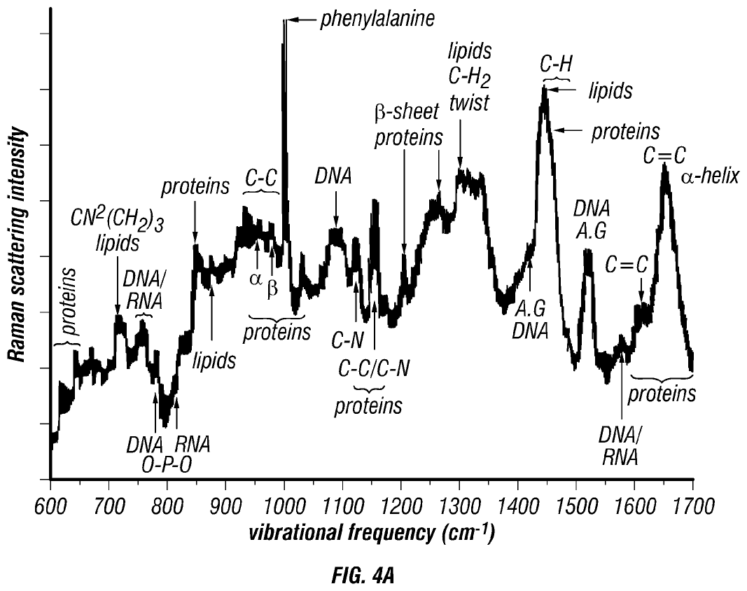

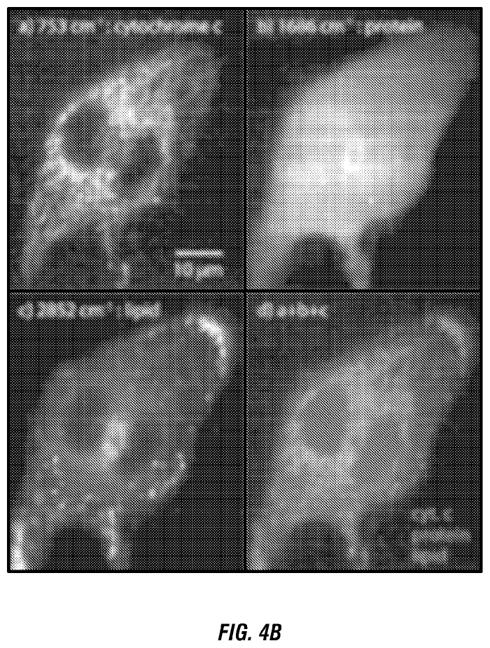

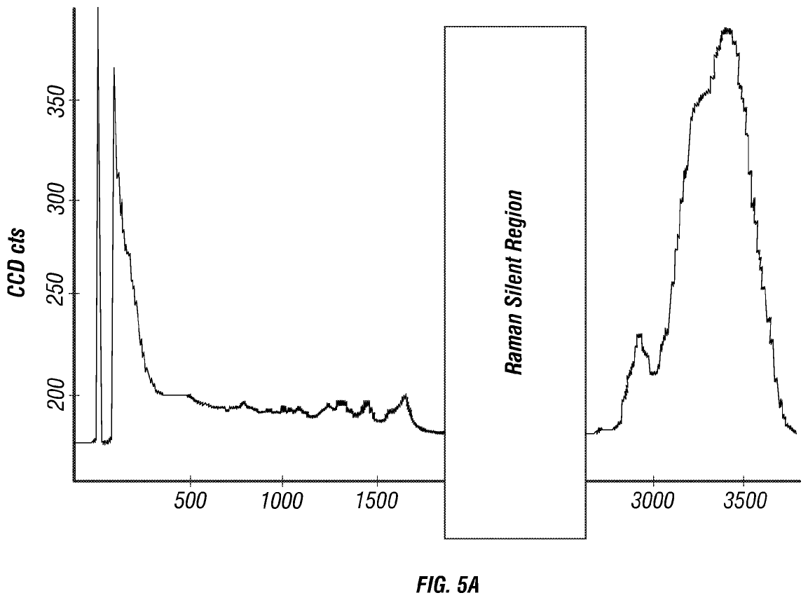

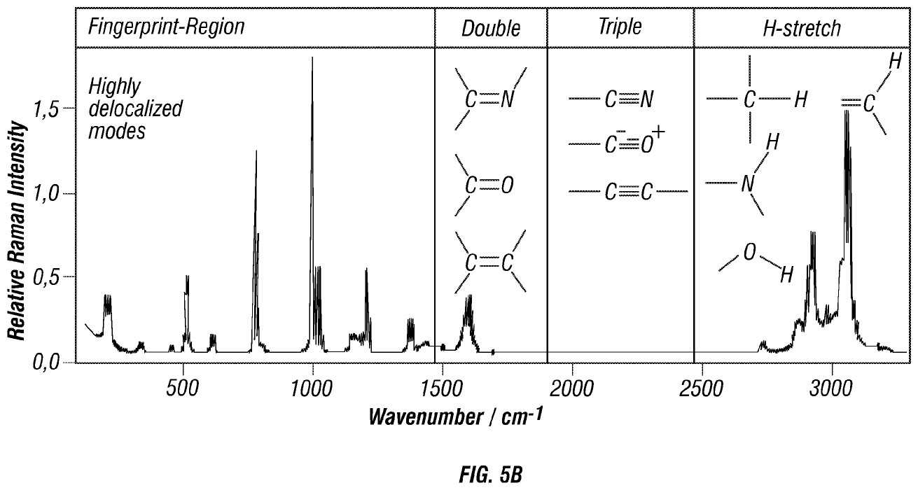

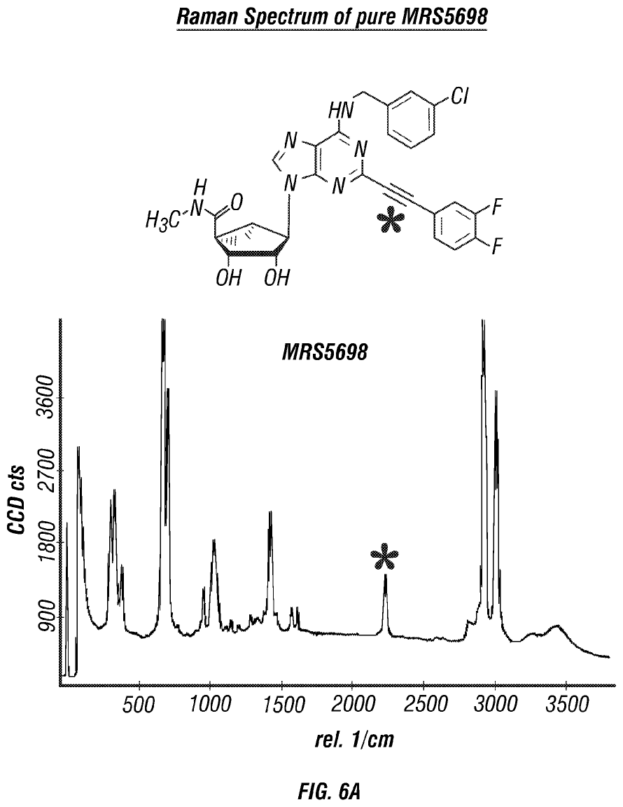

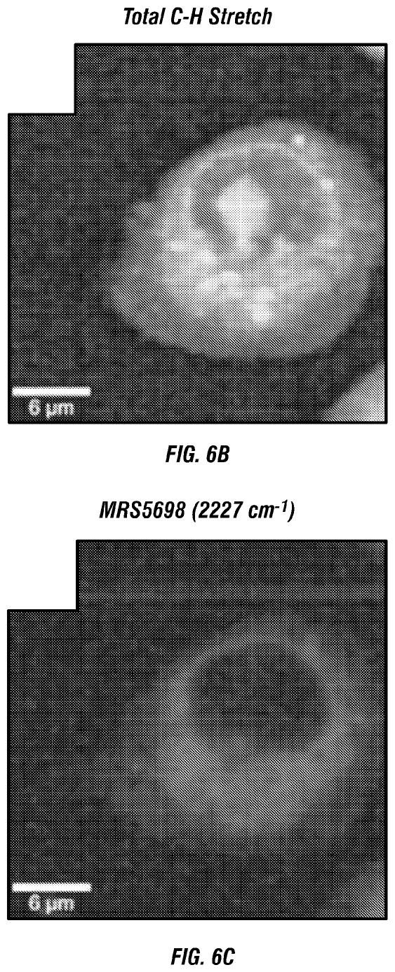

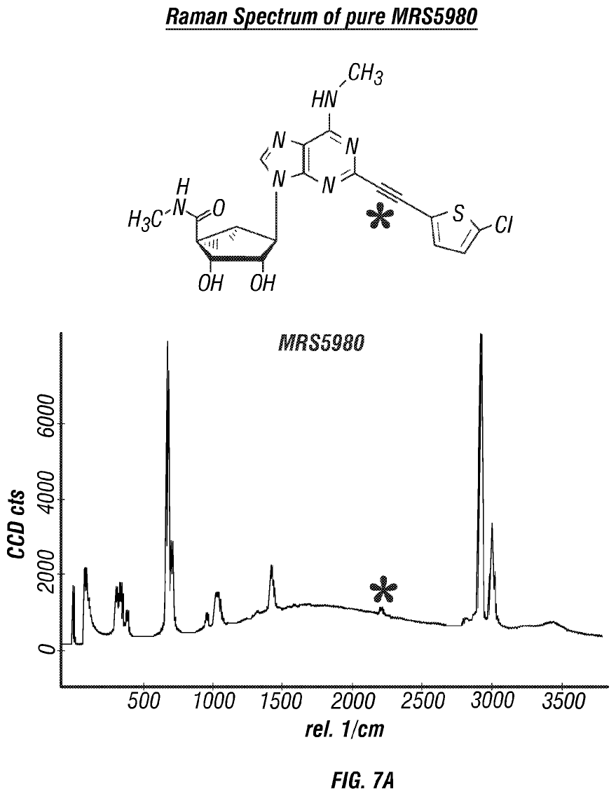

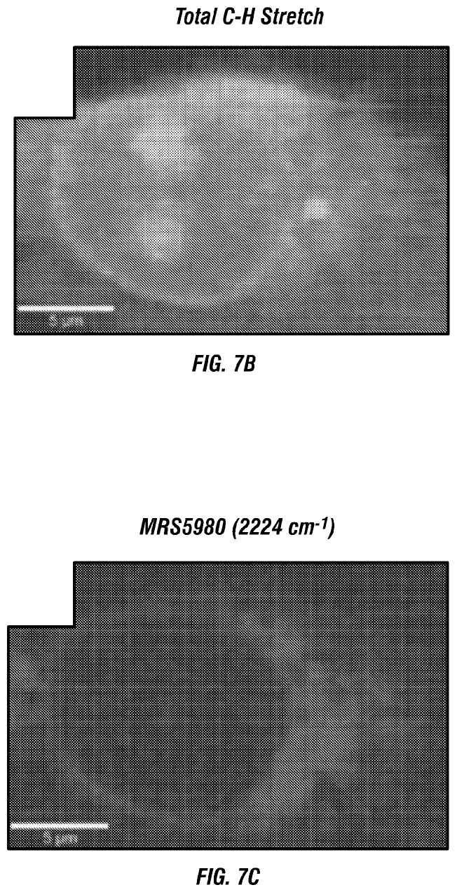

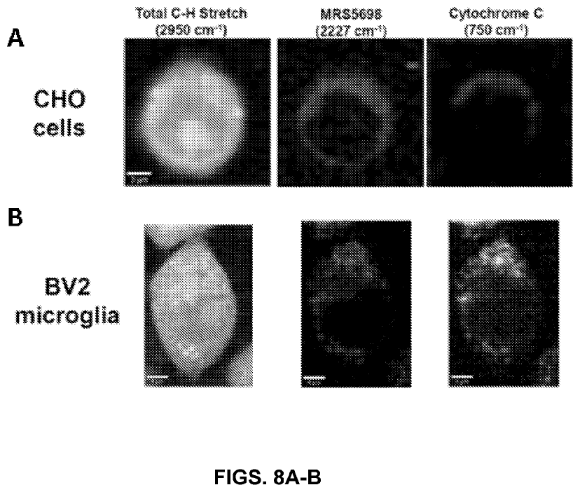

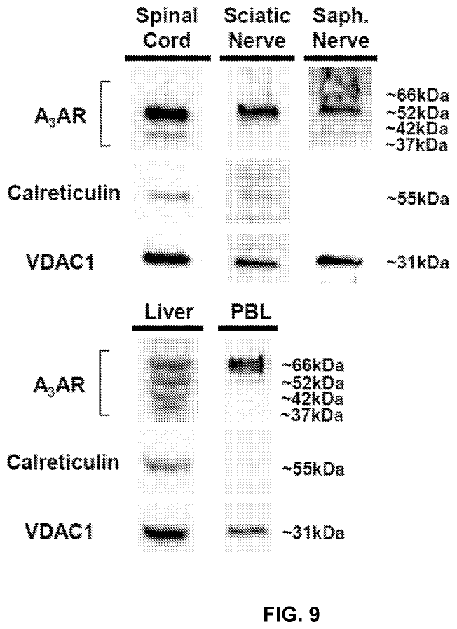

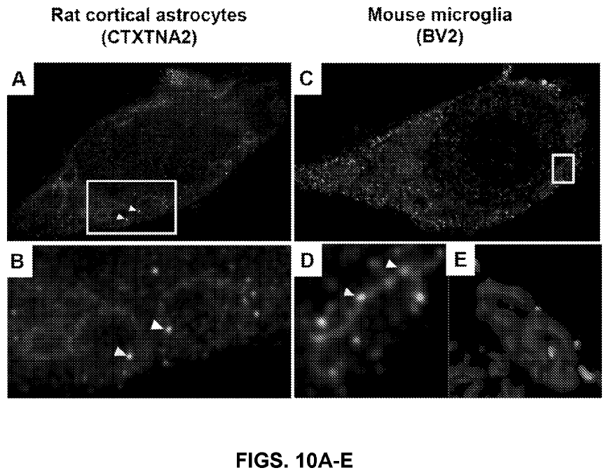

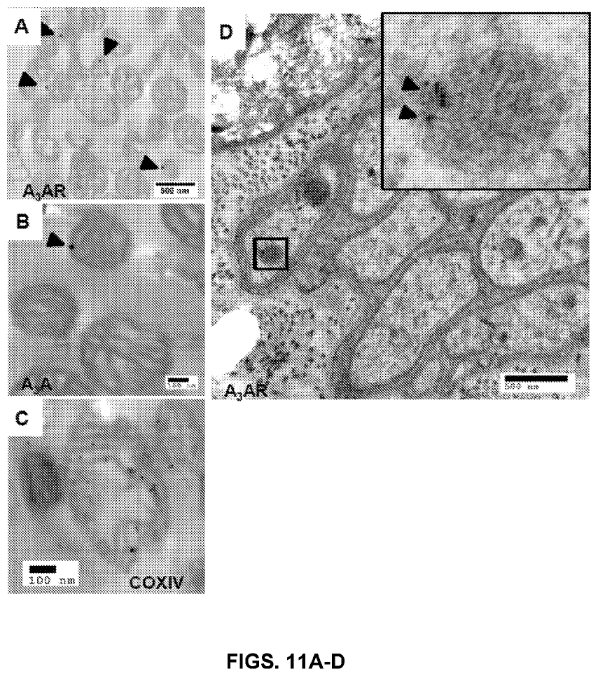

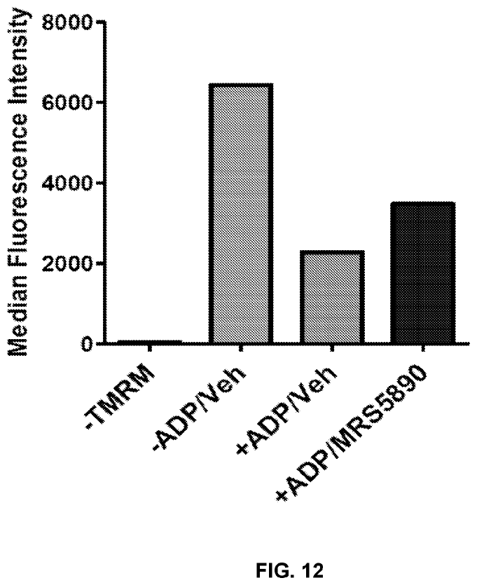

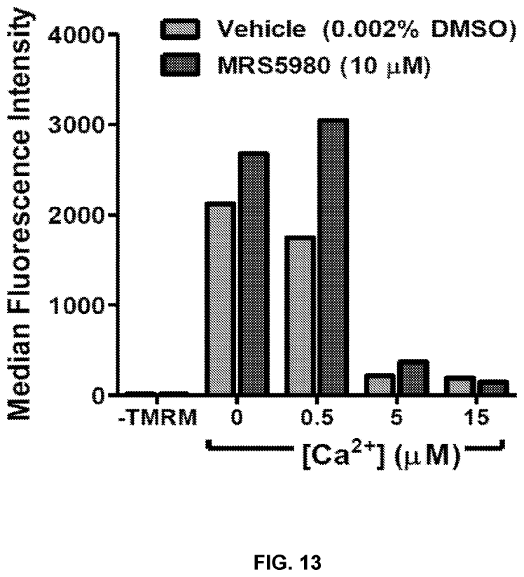

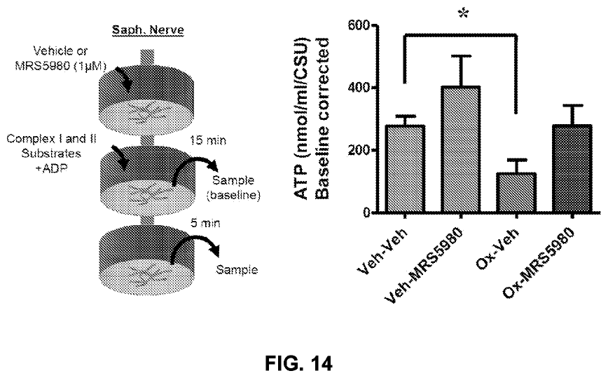

The present disclosure is based on the discovery that treatment with an A3AR agonist during anti-cancer chemotherapy with paclitaxel or oxaliplatin prevents IENF degeneration (FIG. 1) and the associated mitochondrial insult. Thus, A₃AR agonists can be treatments for CIPN-associated neurodegeneration and other neurodegenerative conditions that also involve mitochondrial dysfunction. This and other aspects of the disclosure are set forth in detail below.

### I. NEURODEGNERATIVE DISEASES AND DISORDERS

Neurodegeneration is the umbrella term for the progressive loss of structure or function of neurons, including death of neurons. Many neurological diseases including amyotrophic lateral sclerosis, Parkinson's, Alzheimer's, and Huntington's occur as a result of neurodegenerative processes. Such diseases are incurable, resulting in progressive degeneration and/or death of neuron cells. As research progresses, many similarities appear that relate these diseases to one another on a sub-cellular level. Discovering these similarities offers hope for therapeutic advances that could ameliorate many diseases simultaneously. There are many parallels between different neurodegenerative disorders including atypical protein assemblies as well as induced cell death. There is abundant evidence that for al of these conditions a dysfunction in neuronal mitochondria results in a bioenergetic deficit due to reactive oxygen species and reactive nitrogen species.

A. Alzheimer's Disease

AD is a progressive, neurodegenerative disease characterized by memory loss, language deterioration, impaired visuospatial skills, poor judgment, indifferent attitude, but preserved motor function. AD usually begins after age 65, however, its onset may occur as early as age 40, appearing first as memory decline and, over several years, destroying cognition, personality, and ability to function. Confusion and restlessness may also occur. The type, severity, sequence, and progression of mental changes vary widely. The early symptoms of AD, which include forgetfulness and loss of concentration, can be missed easily because they resemble natural signs of aging. Similar symptoms can also result from fatigue, grief, depression, illness, vision or hearing loss, the use of alcohol or certain medications, or simply the burden of too many details to remember at once.

There is no cure for AD and no way to slow the progression of the disease. For some people in the early or middle stages of the disease, medication such as tacrine may alleviate some cognitive symptoms. Aricept (donepezil) and Exelon (rivastigmine) are reversible acetylcholinesterase inhibitors that are indicated for the treatment of mild to moderate dementia of the Alzheimer's type. Also, some medications may help control behavioral symptoms such as sleeplessness, agitation, wandering, anxiety, and depression. These treatments are aimed at making the patient more comfortable.

AD is a progressive disease. The course of the disease varies from person to person. Some people have the disease only for the last 5 years of life, while others may have it for as many as 20 years. The most common cause of death in AD patients is infection.

The molecular aspect of AD is complicated and not yet fully defined. As stated above, AD is characterized by the formation of amyloid plaques and neurofibrillary tangles in the brain, particularly in the hippocampus which is the center for memory processing. Several molecules contribute to these structures: amyloid β protein (Aβ), presenilin (PS), cholesterol, apolipoprotein E (ApoE), and Tau protein. Of these, Aβ appears to play the central role.

Aβ contains approximately 40 amino acid residues. The 42 and 43 residue forms are much more toxic than the 40 residue form. Aβ is generated from an amyloid precursor protein (APP) by sequential proteolysis. One of the enzymes lacks sequence specificity and thus can generate Aβ of varying (39-43) lengths. The toxic forms of Aβ cause abnormal events such as apoptosis, free radical formation, aggregation and inflammation. Presenilin encodes the protease responsible for cleaving APP into Aβ. There are two forms—PS 1 and PS2. Mutations in PS1, causing production of Aβ42, are the typical cause of early onset AD.

Cholesterol-reducing agents have been alleged to have AD-preventative capabilities, although no definitive evidence has linked elevated cholesterol to increased risk of AD. However, the discovery that Aβ contains a sphingolipid binding domain lends further credence to this theory. Similarly, ApoE, which is involved in the redistribution of cholesterol, is now believed to contribute to AD development. As discussed above, individuals having the ApoE4 allele, which exhibits the least degree of cholesterol efflux from neurons, are more likely to develop AD.

Tau protein, associated with microtubules in normal brain, forms paired helical filaments (PHFs) in AD-affected brains which are the primary constituent of neurofibrillary tangles. Recent evidence suggests that Aβ proteins may cause hyperphosphorylation of Tau proteins, leading to disassociation from microtubules and aggregation into PHFs.

It is well-established that the neurodegeneration of AD involves dysfunction of neuronal mitochondria (Carvalho et al, 2015); ameliorating this dysfunction with A3AR agonist treatment will thus be a potential therapeutic approach for AD.

B. Huntingtin's Disease

Huntington disease, also called Huntington's chorea, chorea major, or HD, is a genetic neurological disorder characterized by abnormal body movements called chorea and a lack of coordination; it also affects a number of mental abilities and some aspects of behavior. In 1993, the gene causing HD was found, making it one of the first inherited genetic disorders for which an accurate test could be performed. The accession number for Huntingtin is NM_002111.

The gene causing the disorder is dominant and may, therefore, be inherited from a single parent. Global incidence varies, from 3 to 7 per 100,000 people of Western European descent, down to 1 per 1,000,000 of Asian and African descent. The onset of physical symptoms in HD occur in a large range around a mean of a person's late forties to early fifties. If symptoms become noticeable before a person is the age of twenty, then their condition is known as Juvenile HD.

A trinucleotide repeat expansion occurs in the Huntingtin gene, which produces mutant Huntingtin protein. The presence of this protein increases the rate of neuron cell death in select areas of the brain, affecting certain neurological functions. The loss of neurons isn't fatal, but complications caused by symptoms reduce life expectancy. There is currently no proven cure, so symptoms are managed with a range of medications and supportive services.

Symptoms increase in severity progressively, but are not often recognised until they reach certain stages. Physical symptoms are usually the first to cause problems and be noticed, but these are accompanied by cognitive and psychiatric ones which aren't often recognized. Almost everyone with HD eventually exhibits all physical symptoms, but cognitive symptoms vary, and so any psychopathological problems caused by these, also vary per individual. The symptoms of juvenile HD differ in that they generally progress faster and are more likely to exhibit rigidity and bradykinesia instead of chorea and often include seizures.

The most characteristic symptoms are jerky, random, and uncontrollable movements called chorea, although sometimes very slow movement and stiffness (bradykinesia, dystonia) can occur instead or in later stages. These abnormal movements are initially exhibited as general lack of coordination, an unsteady gait and slurring of speech. As the disease progresses, any function that requires muscle control is affected, this causes reduced physical stability, abnormal facial expression, impaired speech comprehensibility, and difficulties chewing and swallowing. Eating difficulties commonly cause weight loss. HD has been associated with sleep cycle disturbances, including insomnia and rapid eye movement sleep alterations.

Selective cognitive abilities are progressively impaired, including executive function (planning, cognitive flexibility, abstract thinking, rule acquisition, initiating appropriate actions and inhibiting inappropriate actions), psychomotor function (slowing of thought processes to control muscles), perceptual and spatial skills of self and surrounding environment, selection of correct methods of remembering information (but not actual memory itself), short-term memory, and ability to learn new skills, depending on the pathology of the individual.

Psychopathological symptoms vary more than cognitive and physical ones, and may include anxiety, depression, a reduced display of emotions (blunted affect) and decreased ability to recognize negative expressions like anger, disgust, fear or sadness in others, egocentrism, aggression, and compulsive behavior. The latter can cause, or worsen, hypersexuality and addictions such as alcoholism and gambling.

HD is autosomal dominant, needing only one affected allele from either parent to inherit the disease. Although this generally means there is a one in two chance of inheriting the disorder from an affected parent, the inheritance of HD is more complex due to potential dynamic mutations, where DNA replication does not produce an exact copy of itself. This can cause the number of repeats to change in successive generations. This can mean that a parent with a count close to the threshold, may pass on a gene with a count either side of the threshold. Repeat counts maternally inherited are usually similar, whereas paternally inherited ones tend to increase. This potential increase in repeats in successive generations is known as anticipation. In families where neither parent has HD, new mutations account for truly sporadic cases of the disease. The frequency of these de novo mutations is extremely low.

Homozygous individuals, who carry two mutated genes because both parents passed on one, are rare. While HD seemed to be the first disease for which homozygotes did not differ in clinical expression or course from typical heterozygotes, more recent analysis suggest that homozygosity affects the phenotype and the rate of disease progression though it does not alter the age of onset suggesting that the mechanisms underlying the onset and the progression are different.

Huntingtin protein is variable in its structure as there are many polymorphisms of the gene which can lead to variable numbers of glutamine residues present in the protein. In its wild-type (normal) form, it contains 6-35 glutamine residues; however, in individuals affected by HD, it contains between 36-155 glutamine residues. Huntingtin has a predicted mass of −350 kDa, however, this varies and is largely dependent on the number of glutamine residues in the protein. Normal huntingtin is generally accepted to be 3144 amino acids in size.

Two transcriptional pathways are more extensively implicated in HD—the CBP/p300 and Sp1 pathways—and these are transcription factors whose functions are vital for the expression of many genes. The postulated relationship between CBP and HD stems from studies showing that CBP is found in poly glutamine aggregates (see Kazantsev et al., 1999). Consequently, it was demonstrated that huntingtin and CBP interact via their polyglutamine stretches, that huntingtin with an expanded polyglutamine tract interferes with CBP-activated gene expression, and that overexpression of CBP rescued polyglutamine-induced toxicity in cultured cells (Nucifora et al, 2001; Steffan et al, 2001). Mutant huntingtin was also shown to interact with the acetyltransferase domain of CBP and inhibit the acetyltransferase activity of CBP, p300, and the p300/CBP-associated factor P/CAF (Steffan et al, 2001).

These observations prompted a hypothesis whereby the pathogenic process was linked to the state of histone acetylation; specifically, mutant huntingtin induced a state of decreased histone acetylation and thus altered gene expression. Support for this hypothesis was obtained in a Drosophila HD model expressing an N-terminal fragment of huntingtin with an expanded polyglutamine tract in the eye. Administration of inhibitors of histone deacetylase arrested the neurodegeneration and lethality (Steffan et al, 2001). Protective effects of HDAC inhibitors have been reported for other polyglutamine disorders, prompting the concept that at least some of the observed effects in polyglutamine disorders are due to alterations in histone acetylation (Hughes 2002). Studies published in 2002 revealed that the N-terminal fragment of huntingtin and intact huntingtin interact with Sp1 (Dunah et al, 2002; Li et al, 2002), a transcriptional activator that binds to upstream GC-nch elements in certain promoters. It is the glutamine-rich transactivation domain of Sp1 that selectively binds and directs core components of the general transcriptional complex such as TFIID, TBP and other TBP-associated factors to Sp1-dependent sites of transcription. In vitro transcription studies have gone on to show that in addition to targeting Sp1, mutant huntingtin targets TFIID and TFIIF, members of the core transcriptional complex (Zhai et al 2005). Mutant huntingtin was shown to interact with the RAP30 subunit of TFIIF. Notably, overexpression of RAP30 alleviated both mutant huntingtin-induced toxicity and transcriptional repression of the dopamine D2 receptor gene. These results indicate that mutant huntingtin may interfere with multiple components of the transcription machinery.

There is no treatment to fully arrest the progression of the disease, but symptoms can be reduced or alleviated through the use of medication and care methods. Huntington mice models exposed to better husbandry techniques, especially better access to food and water, lived much longer than mice that were not well cared for.

Standard treatments to alleviate emotional symptoms include the use of antidepressants and sedatives, with antipsychotics (in low doses) for psychotic symptoms. Speech therapy helps by improving speech and swallowing methods; this therapy is more effective if started early on, as the ability to learn is reduced as the disease progresses. A two-year pilot study, of intensive speech, pyschiatric and physical therapy, applied to inpatient rehabilitation, showed motor decline was greatly reduced.

Nutrition is an important part of treatment; most third and fourth stage HD sufferers need two to three times the calories of the average person to maintain body weight. Healthier foods in pre-symptomatic and earlier stages may slow down the onset and progression of the disease. High calorie intake in pre-symptomatic and earlier stages has been shown to speed up the onset and reduce IQ level. Thickening agent can be added to drinks as swallowing becomes more difficult, as thicker fluids are easier and safer to swallow. The option of using a stomach PEG is available when eating becomes too hazardous or uncomfortable; this greatly reduces the chances of aspiration of food, and the subsequent increased risk of pneumonia, and increases the amount of nutrients and calories that can be ingested.

EPA, an Omega-3 fatty acid, may slow and possibly reverse the progression of the disease. As of April 2008, it is in FDA clinical trial as ethyl-EPA, (brand name Miraxion), for prescription use. Clinical trials utilise 2 grams per day of EPA. In the United States, it is available over the counter in lower concentrations in Omega-3 and fish oil supplements.

It is well-established that the neurodegeneration of HD involves dysfunction of neuronal mitochondria (Carvalho et al, 2015); ameliorating this dysfunction with A3AR agonist treatment will thus be a potential therapeutic approach for HD.

C. Parkinson's Disease

Parkinson's disease (PD) is a degenerative disorder of the central nervous system. The motor symptoms of Parkinson's disease result from the death of dopamine-generating cells in the substantia nigra, a region of the midbrain; the cause of cell-death is unknown. Early in the course of the disease, the most obvious symptoms are movement-related, including shaking, rigidity, slowness of movement and difficulty with walking and gait. Later, cognitive and behavioural problems may arise, with dementia commonly occurring in the advanced stages of the disease. Other symptoms include sensory, sleep and emotional problems. PD is more common in the elderly with most cases occurring after the age of 50.

The main motor symptoms are collectively called parkinsonism, or a “parkinsonian syndrome.” Parkinson's disease is often defined as a parkinsonian syndrome that is idiopathic (having no known cause), although some atypical cases have a genetic origin. Many risk and protective factors have been investigated: the clearest evidence is for an increased risk of PD in people exposed to certain pesticides and a reduced risk in tobacco smokers. The pathology of the disease is characterized by the accumulation of a protein called alpha-synuclein into inclusions called Lewy bodies in neurons, and from insufficient formation and activity of dopamine produced in certain neurons within parts of the midbrain. Lewy bodies are the pathological hallmark of the idiopathic disorder and the distribution of the Lewy bodies throughout the Parkinsonian brain varies from one individual to another. The anatomical distribution of the Lewy body is often directily related to the expression and degree of the clinical symptoms of each individual. Diagnosis of typical cases is mainly based on symptoms, with tests such as neuroimaging being used for confirmation.

Modern treatments are effective at managing the early motor symptoms of the disease, mainly through the use of levodopa and dopamine agonists. As the disease progresses and dopamine neurons continue to be lost, a point eventually arrives at which these drugs become ineffective at treating the symptoms and at the same time produce a complication called dyskinesia, marked by involuntary writhing movements. Diet and some forms of rehabilitation have shown some effectiveness at alleviating symptoms. Surgery and deep brain stimulation have been used to reduce motor symptoms as a last resort in severe cases where drugs are ineffective. Research directions include a search of new animal models of the disease and investigations of the potential usefulness of gene therapy, stem cell transplants and neuroprotective agents. Medications to treat non-movement-related symptoms of PD, such as sleep disturbances and emotional problems, also exist.

The term parkinsonism is used for a motor syndrome whose main symptoms are tremor at rest, stiffness, slowing of movement and postural instability. Parkinsonian syndromes can be divided into four subtypes according to their origin: primary or idiopathic, secondary or acquired, hereditary parkinsonism, and parkinson plus syndromes or multiple system degeneration. Parkinson's disease is the most common form of parkinsonism and is usually defined as “primary” parkinsonism, meaning parkinsonism with no external identifiable cause. In recent years several genes that are directly related to some cases of Parkinson's disease have been discovered. As much as this can go against the definition of Parkinson's disease as an idiopathic illness, genetic parkinsonism disorders with a similar clinical course to PD are generally included under the Parkinson's disease label. The terms “familial Parkinson's disease” and “sporadic Parkinson's disease” can be used to differentiate genetic from truly idiopathic forms of the disease.

PD is usually classified as a movement disorder, although it also gives rise to several non-motor types of symptoms such as sensory deficits, cognitive difficulties or sleep problems. Parkinson plus diseases are primary parkinsonisms which present additional features. They include multiple system atrophy, progressive supranuclear palsy, corticobasal degeneration and dementia with Lewy bodies.

In terms of pathophysiology, PD is considered a synucleinopathy due to an abnormal accumulation of alpha-synuclein protein in the brain in the form of Lewy bodies, as opposed to other diseases such as Alzheimer's disease where the brain accumulates tau protein in the form of neurofibrillary tangles. Nevertheless, there is clinical and pathological overlap between tauopathies and synucleinopathies. The most typical symptom of Alzheimer's disease, dementia, occurs in advanced stages of PD, while it is common to find neurofibrillary tangles in brains affected by PD.

Dementia with Lewy bodies (DLB) is another synucleinopathy that has similarities with PD, and especially with the subset of PD cases with dementia. However the relationship between PD and DLB is complex and still has to be clarified. They may represent parts of a continuum or they may be separate diseases.

Four motor symptoms are considered cardinal in PD: tremor, rigidity, slowness of movement, and postural instability. Tremor is the most apparent and well-known symptom. It is the most common; though around 30% of individuals with PD do not have tremor at disease onset, most develop it as the disease progresses. It is usually a rest tremor: maximal when the limb is at rest and disappearing with voluntary movement and sleep. It affects to a greater extent the most distal part of the limb and at onset typically appears in only a single arm or leg, becoming bilateral later. Frequency of PD tremor is between 4 and 6 hertz (cycles per second). A feature of tremor is “pill-rolling,” a term used to describe the tendency of the index finger of the hand to get into contact with the thumb and perform together a circular movement. The term derives from the similarity between the movement in PD patients and the earlier pharmaceutical technique of manually making pills.

Bradykinesia (slowness of movement) is another characteristic feature of PD, and is associated with difficulties along the whole course of the movement process, from planning to initiation and finally execution of a movement. Performance of sequential and simultaneous movement is hindered. Bradykinesia is the most disabling symptom in the early stages of the disease. Initial manifestations are problems when performing daily tasks which require fine motor control such as writing, sewing or getting dressed. Clinical evaluation is based in similar tasks such as alternating movements between both hands and both feet. Bradykinesia is not equal for all movements or times. It is modified by the activity or emotional state of the subject, to the point that some patients are barely able to walk yet can still ride a bicycle. Generally patients have less difficulty when some sort of external cue is provided.

Rigidity is stiffness and resistance to limb movement caused by increased muscle tone, an excessive and continuous contraction of muscles. In parkinsonism the rigidity can be uniform (lead-pipe rigidity) or ratchety (cogwheel rigidity). The combination of tremor and increased tone is considered to be at the origin of cogwheel rigidity. Rigidity may be associated with joint pain; such pain being a frequent initial manifestation of the disease. In early stages of Parkinson's disease, rigidity is often asymmetrical and it tends to affect the neck and shoulder muscles prior to the muscles of the face and extremities. With the progression of the disease, rigidity typically affects the whole body and reduces the ability to move.

Postural instability is typical in the late stages of the disease, leading to impaired balance and frequent falls, and secondarily to bone fractures. Instability is often absent in the initial stages, especially in younger people. Up to 40% of the patients may experience falls and around 10% may have falls weekly, with number of falls being related to the severity of PD.

Other recognized motor signs and symptoms include gait and posture disturbances such as festination (rapid shuffling steps and a forward-flexed posture when walking), speech and swallowing disturbances including voice disorders, mask-like face expression or small handwriting, although the range of possible motor problems that can appear is large.

Parkinson's disease can cause neuropsychiatric disturbances which can range from mild to severe. This includes disorders of speech, cognition, mood, behaviour, and thought. Cognitive disturbances can occur in the initial stages of the disease and sometimes prior to diagnosis, and increase in prevalence with duration of the disease. The most common cognitive deficit in affected individuals is executive dysfunction, which can include problems with planning, cognitive flexibility, abstract thinking, rule acquisition, initiating appropriate actions and inhibiting inappropriate actions, and selecting relevant sensory information. Fluctuations in attention and slowed cognitive speed are among other cognitive difficulties. Memory is affected, specifically in recalling learned information. Nevertheless, improvement appears when recall is aided by cues. Visuospatial difficulties are also part of the disease, seen for example when the individual is asked to perform tests of facial recognition and perception of the orientation of drawn lines.

A person with PD has two to six times the risk of suffering dementia compared to the general population. The prevalence of dementia increases with duration of the disease. Dementia is associated with a reduced quality of life in people with PD and their caregivers, increased mortality, and a higher probability of needing nursing home care. Behavior and mood alterations are more common in PD without cognitive impairment than in the general population, and are usually present in PD with dementia. The most frequent mood difficulties are depression, apathy and anxiety. Impulse control behaviors such as medication overuse and craving, binge eating, hypersexuality, or pathological gambling can appear in PD and have been related to the medications used to manage the disease. Psychotic symptoms-hallucinations or delusions-occur in 4% of patients, and it is assumed that the main precipitant of psychotic phenomena in Parkinson's disease is dopaminergic excess secondary to treatment; it therefore becomes more common with increasing age and levodopa intake.

In addition to cognitive and motor symptoms, PD can impair other body functions. Sleep problems are a feature of the disease and can be worsened by medications. Symptoms can manifest in daytime drowsiness, disturbances in REM sleep, or insomnia. Alterations in the autonomic nervous system can lead to orthostatic hypotension (low blood pressure upon standing), oily skin and excessive sweating, urinary incontinence and altered sexual function. Constipation and gastric dysmotility can be severe enough to cause discomfort and even endanger health. PD is related to several eye and vision abnormalities such as decreased blink rate, dry eyes, deficient ocular pursuit (eye tracking) and saccadic movements (fast automatic movements of both eyes in the same direction), difficulties in directing gaze upward, and blurred or double vision. Changes in perception may include an impaired sense of smell, sensation of pain and paresthesia (skin tingling and numbness). All of these symptoms can occur years before diagnosis of the disease.

A physician will diagnose PD from the medical history and a neurological examination. There is no lab test that will clearly identify the disease, but brain scans are sometimes used to rule out disorders that could give rise to similar symptoms. Patients may be given levodopa and resulting relief of motor impairment tends to confirm diagnosis. The finding of Lewy bodies in the midbrain on autopsy is usually considered proof that the patient suffered from PD. The progress of the illness over time may reveal it is not PD, and some authorities recommend that the diagnosis be periodically reviewed.

Other causes that can secondarily produce a parkinsonian syndrome are Alzheimer's disease, multiple cerebral infarction and drug-induced parkinsonism. Parkinson plus syndromes such as progressive supranuclear palsy and multiple system atrophy must be ruled out. Anti-Parkinson's medications are typically less effective at controlling symptoms in Parkinson plus syndromes. Faster progression rates, early cognitive dysfunction or postural instability, minimal tremor or symmetry at onset may indicate a Parkinson plus disease rather than PD itself. Genetic forms are usually classified as PD, although the terms familial Parkinson's disease and familial parkinsonism are used for disease entities with an autosomal dominant or recessive pattern of inheritance.

Computed tomography (CT) and magnetic resonance imaging (MRI) brain scans of people with PD usually appear normal. These techniques are nevertheless useful to rule out other diseases that can be secondary causes of parkinsonism, such as basal ganglia tumors, vascular pathology and hydrocephalus. A specific technique of MRI, diffusion MRI, has been reported to be useful at discriminating between typical and atypical parkinsonism, although its exact diagnostic value is still under investigation. Dopaminergic function in the basal ganglia can be measured with different PET and SPECT radiotracers. Examples are loflupane (1231) (trade name DaTSCAN) and iometopane (Dopascan) for SPECT or fludeoxy glucose (18F) for PET. A pattern of reduced dopaminergic activity in the basal ganglia can aid in diagnosing PD.

There is no cure for PD, but medications, surgery and multidisciplinary management can provide relief from the symptoms. The main families of drugs useful for treating motor symptoms are levodopa (usually combined with a dopa decarboxylase inhibitor or COMT inhibitor), dopamine agonists and MAO-B inhibitors. The stage of the disease determines which group is most useful. Two stages are usually distinguished: an initial stage in which the individual with PD has already developed some disability for which he needs pharmacological treatment, then a second stage in which an individual develops motor complications related to levodopa usage. Treatment in the initial stage aims for an optimal tradeoff between good symptom control and side-effects resulting from enhancement of dopaminergic function. The start of levodopa (or L-DOPA) treatment may be delayed by using other medications such as MAO-B inhibitors and dopamine agonists, in the hope of delaying the onset of dyskinesias. In the second stage the aim is to reduce symptoms while controlling fluctuations of the response to medication. Sudden withdrawals from medication or overuse have to be managed. When medications are not enough to control symptoms, surgery and deep brain stimulation can be of use. In the final stages of the disease, palliative care is provided to enhance quality of life.

Levodopa has been the most widely used treatment for over 30 years. L-DOPA is converted into dopamine in the dopaminergic neurons by dopa decarboxylase. Since motor symptoms are produced by a lack of dopamine in the substantia nigra, the administration of L-DOPA temporarily diminishes the motor symptoms. Only 5-10% of L-DOPA crosses the blood-brain barrier. The remainder is often metabolized to dopamine elsewhere, causing a variety of side effects including nausea, dyskinesias and joint stiffness. Carbidopa and benserazide are peripheral dopa decarboxylase inhibitors, which help to prevent the metabolism of L-DOPA before it reaches the dopaminergic neurons, therefore reducing side effects and increasing bioavailability. They are generally given as combination preparations with levodopa. Existing preparations are carbidopa/levodopa (co-careldopa) and benserazide/levodopa (co-beneldopa). Levodopa has been related to dopamine dysregulation syndrome, which is a compulsive overuse of the medication, and punding. There are controlled release versions of levodopa in the form intravenous and intestinal infusions that spread out the effect of the medication. These slow-release levodopa preparations have not shown an increased control of motor symptoms or motor complications when compared to immediate release preparations.

Tolcapone inhibits the COMT enzyme, which degrades dopamine, thereby prolonging the effects of levodopa. It has been used to complement levodopa; however, its usefulness is limited by possible side effects such as liver damage. A similarly effective drug, entacapone, has not been shown to cause significant alterations of liver function. Licensed preparations of entacapone contain entacapone alone or in combination with carbidopa and levodopa.

Levodopa preparations lead in the long term to the development of motor complications characterized by involuntary movements called dyskinesias and fluctuations in the response to medication. When this occurs a person with PD can change from phases with good response to medication and few symptoms (“on” state), to phases with no response to medication and significant motor symptoms (“off” state). For this reason, levodopa doses are kept as low as possible while maintaining functionality. Delaying the initiation of therapy with levodopa by using alternatives (dopamine agonists and MAO-B inhibitors) is common practice. A former strategy to reduce motor complications was to withdraw L-DOPA medication for some time. This is discouraged now, since it can bring dangerous side effects such as neuroleptic malignant syndrome. Most people with PD will eventually need levodopa and later develop motor side effects.

Several dopamine agonists that bind to dopaminergic post-synaptic receptors in the brain have similar effects to levodopa. These were initially used for individuals experiencing on-off fluctuations and dyskinesias as a complementary therapy to levodopa; they are now mainly used on their own as an initial therapy for motor symptoms with the aim of delaying motor complications. When used in late PD they are useful at reducing the off periods. Dopamine agonists include bromocriptine, pergolide, pramipexole, ropinirole, piribedil, cabergoline, apomorphine and lisuride.

Dopamine agonists produce significant, although usually mild, side effects including drowsiness, hallucinations, insomnia, nausea and constipation. Sometimes side effects appear even at a minimal clinically effective dose, leading the physician to search for a different drug. Compared with levodopa, dopamine agonists may delay motor complications of medication use but are less effective at controlling symptoms. Nevertheless, they are usually effective enough to manage symptoms in the initial years. They tend to be more expensive than levodopa. Dyskinesias due to dopamine agonists are rare in younger people who have PD, but along with other side effects, become more common with age at onset. Thus dopamine agonists are the preferred initial treatment for earlier onset, as opposed to levodopa in later onset. Agonists have been related to a impulse control disorders (such as compulsive sexual activity and eating, and pathological gambling and shopping) even more strongly than levodopa.

Apomorphine, a non-orally administered dopamine agonist, may be used to reduce off periods and dyskinesia in late PD. It is administered by intermittent injections or continuous subcutaneous infusions. Since secondary effects such as confusion and hallucinations are common, individuals receiving apomorphine treatment should be closely monitored. Two dopamine agonists that are administered through skin patches (lisuride and rotigotine) have been recently found to be useful for patients in initial stages and preliminary positive results has been published on the control of off states in patients in the advanced state.

MAO-B inhibitors (selegiline and rasagiline) increase the level of dopamine in the basal ganglia by blocking its metabolism. They inhibit monoamine oxidase-B (MAO-B) which breaks down dopamine secreted by the dopaminergic neurons. The reduction in MAO-B activity results in increased L-DOPA in the striatum. Like dopamine agonists, MAO-B inhibitors used as monotherapy improve motor symptoms and delay the need for levodopa in early disease, but produce more adverse effects and are less effective than levodopa. There are few studies of their effectiveness in the advanced stage, although results suggest that they are useful to reduce fluctuations between on and off periods. An initial study indicated that selegiline in combination with levodopa increased the risk of death, but this was later disproven.

Other drugs such as amantadine and anticholinergics may be useful as treatment of motor symptoms. However, the evidence supporting them lacks quality, so they are not first choice treatments. In addition to motor symptoms, PD is accompanied by a diverse range of symptoms. A number of drugs have been used to treat some of these problems. Examples are the use of clozapine for psychosis, cholinesterase inhibitors for dementia, and modafinil for daytime sleepiness. A 2010 meta-analysis found that non-steroidal anti-inflammatory drugs (apart from acetaminophen and aspirin), have been associated with at least a 15 percent (higher in long-term and regular users) reduction of incidence of the development of Parkinson's disease.

Placement of an electrode into the brain. The head is stabilised in a frame for stereotactic surgery. Treating motor symptoms with surgery was once a common practice, but since the discovery of levodopa, the number of operations declined. Studies in the past few decades have led to great improvements in surgical techniques, so that surgery is again being used in people with advanced PD for whom drug therapy is no longer sufficient. Surgery for PD can be divided in two main groups: lesional and deep brain stimulation (DBS). Target areas for DBS or lesions include the thalamus, the globus pallidus or the subthalamic nucleus. Deep brain stimulation (DBS) is the most commonly used surgical treatment. It involves the implantation of a medical device called a brain pacemaker, which sends electrical impulses to specific parts of the brain. DBS is recommended for people who have PD who suffer from motor fluctuations and tremor inadequately controlled by medication, or to those who are intolerant to medication, as long as they do not have severe neuropsychiatric problems. Other, less common, surgical therapies involve the formation of lesions in specific subcortical areas (a technique known as pallidotomy in the case of the lesion being produced in the globus pallidus).

There is some evidence that speech or mobility problems can improve with rehabilitation, although studies are scarce and of low quality. Regular physical exercise with or without physiotherapy can be beneficial to maintain and improve mobility, flexibility, strength, gait speed, and quality of life. However, when an exercise program is performed under the supervision of a physiotherapist, there are more improvements in motor symptoms, mental and emotional functions, daily living activities, and quality of life compared to a self-supervised exercise program at home. In terms of improving flexibility and range of motion for patients experiencing rigidity, generalized relaxation techniques such as gentle rocking have been found to decrease excessive muscle tension. Other effective techniques to promote relaxation include slow rotational movements of the extremities and trunk, rhythmic initiation, diaphragmatic breathing, and meditation techniques. As for gait and addressing the challenges associated with the disease such as hypokinesia (slowness of movement), shuffling and decreased arm swing; physiotherapists have a variety of strategies to improve functional mobility and safety. Areas of interest with respect to gait during rehabilitation programs focus on but are not limited to improving gait speed, base of support, stride length, trunk and arm swing movement. Strategies include utilizing assistive equipment (pole walking and treadmill walking), verbal cueing (manual, visual and auditory), exercises (marching and PNF patterns) and altering environments (surfaces, inputs, open vs. closed). Strengthening exercises have shown improvements in strength and motor function for patients with primary muscular weakness and weakness related to inactivity with mild to moderate Parkinson's disease. However, reports show a significant interaction between strength and the time the medications was taken. Therefore, it is recommended that patients should perform exercises 45 minutes to one hour after medications, when the patient is at their best. Also, due to the forward flexed posture, and respiratory dysfunctions in advanced PD, deep diaphragmatic breathing exercises are beneficial in improving chest wall mobility and vital capacity. Exercise may improve constipation.

Palliative care is often required in the final stages of the disease when all other treatment strategies have become ineffective. The aim of palliative care is to maximize the quality of life for the person with the disease and those surrounding him or her. Some central issues of palliative care are: care in the community while adequate care can be given there, reducing or withdrawing drug intake to reduce drug side effects, preventing pressure ulcers by management of pressure areas of inactive patients, and facilitating end-of-life decisions for the patient as well as involved friends and relatives.

It is well-established that the neurodegeneration of PD involves dysfunction of neuronal mitochondria (Carvalho et al, 2015); ameliorating this dysfunction with A3AR agonist treatment will thus be a potential therapeutic approach for PD.

D. Amyotrophic Lateral Sclerosis

Amyotrophic lateral sclerosis (ALS), sometimes called Lou Gehrig's Disease, affects as many as 20,000 Americans at any given time, with 5,000 new cases being diagnosed in the United States each year. ALS affects people of all races and ethnic backgrounds. Men are about 1.5 times more likely than women to be diagnosed with the disease. ALS strikes in the prime of life, with people most commonly diagnosed between the ages of 40 and 70. However, it is possible for individuals to be diagnosed at younger and older ages. About 90-95% of ALS cases occur at random, meaning that individuals do not have a family history of the disease and other family members are not at increased risk for contracting the disease. In about 5-10% of ALS cases there is a family history of the disease.

ALS is a progressive neurological disease that attacks neurons that control voluntary muscles. Motor neurons, which are lost in ALS, are specialized nerve cells located in the brain, brainstem, and spinal cord. These neurons serve as connections from the nervous system to the muscles in the body, and their function is necessary for normal muscle movement. ALS causes motor neurons in both the brain and spinal cord to degenerate, and thus lose the ability to initiate and send messages to the muscles in the body. When the muscles become unable to function, they gradually atrophy and twitch. ALS can begin with very subtle symptoms such as weakness in affected muscles. Where this weakness first appears differs for different people, but the weakness and atrophy spread to other parts of the body as the disease progresses.

Initial symptoms may affect only one leg or arm, causing awkwardness and stumbling when walking or running. Subjects also may suffer difficulty lifting objects or with tasks that require manual dexterity. Eventually, the individual will not be able to stand or walk or use hands and arms to perform activities of daily living. In later stages of the disease, when the muscles in the diaphragm and chest wall become too weak, patients require a ventilator to breathe. Most people with ALS die from respiratory failure, usually 3 to 5 years after being diagnosed; however, some people survive 10 or more years after diagnosis.

Perhaps the most tragic irony of ALS is that it does not impair a person's mind, as the disease affects only the motor neurons. Personality, intelligence, memory, and self-awareness are not affected, nor are the senses of sight, smell, touch, hearing, and taste. Yet at the same time, ALS causes dramatic defects in an individual's ability to speak loudly and clearly, and eventually, completely prevents speaking and vocalizing. Early speech-related symptoms include nasal speech quality, difficulty pronouncing words, and difficulty with conversation. As muscles for breathing weaken, it becomes difficult for patients to speak loud enough to be understood and, eventually, extensive muscle atrophy eliminates the ability to speak altogether. Patients also experience difficulty chewing and swallowing, which increase over time to the point that a feeding tube is required.

It is well-established that the neurodegeneration of ALS involves dysfunction of neuronal mitochondria (Cozzolino et al, 2015); ameliorating this dysfunction with A3AR agonist treatment will thus be a potential therapeutic approach for ALS.

E. Leber's Optic Neuropathy

Leber's hereditary optic neuropathy (LHON) or Leber optic atrophy is a mitochondrially inherited (transmitted from mother to offspring) degeneration of retinal ganglion cells (RGCs) and their axons that leads to an acute or subacute loss of central vision; this affects predominantly young adult males. LHON is only transmitted through the mother, as it is primarily due to mutations in the mitochondrial (not nuclear) genome, and only the egg contributes mitochondria to the embryo. LHON is usually due to one of three pathogenic mitochondrial DNA (mtDNA) point mutations. These mutations are at nucleotide positions 11778 G to A, 3460 G to A and 14484 T to C, respectively in the ND4, ND1 and ND6 subunit genes of complex I of the oxidative phosphorylation chain in mitochondria. Men cannot pass on the disease to their offspring.

This disease was first described by the German ophthalmologist Theodor Leber (1840-1917) in 1871. This disease was initially thought to be X linked but was subsequently shown to be mitochondrial. The nature of the causative mutation was first identified in 1988 by Wallace et al. who discovered the guanine (G) to adenosine (A) mutation at nucleotide position 11778 in nine families. This mutation converts a highly conserved arginine to histidine at codon 340 in the NADH dehydrogenase subunit 4 of complex I of the mitochondrial respiratory chain. The other two mutations known to cause this condition were identified in 1991 (G to A point mutation at nucleotide position 3460) and 1992 (thymidine (T) to cytosine (C) mutation at nucleotide 14484). These three mutations account for over 95% of cases: the 11778 mutation accounts for 50-70% of cases, the 14484 mutation for 10-15% and the 3460 mutation for 8-25%.

Clinically, there is an acute onset of visual loss, first in one eye, and then a few weeks to months later in the other. Onset is usually young adulthood, but age range at onset from 7-75 is reported. The age of onset is slightly higher in females (range 19-55 years: mean 31.3 years) than males (range 15-53 years: mean 24.3). The male to female ratio varies between mutations: 3:1 for 3460 G>A, 6:1 for 11778 G>A and 8:1 for 14484 T>C.

This typically evolves to very severe optic atrophy and permanent decrease of visual acuity. Both eyes become affected either simultaneously (25% of cases) or sequentially (75% of cases) with a median inter-eye delay of 8 weeks. Rarely only one eye may be affected. In the acute stage, lasting a few weeks, the affected eye demonstrates an edematous appearance of the nerve fiber layer especially in the arcuate bundles and enlarged or telangectatic and tortuous peripapillary⁷ vessels (microangiopathy). The main features are seen on fundus examination, just before or subsequent to the onset of visual loss. A pupillary defect may be visible in the acute stage as well. Examination reveals decreased visual acuity, loss of color vision and a cecocentral scotoma on visual field examination.

“LHON Plus” is a name given to rare strains of the disorder with eye disease together with other conditions. The symptoms of this higher form of the disease include loss of the brain's ability to control the movement of muscles, tremors, and cardiac arrhythmia. Many cases of LHON plus have been comparable to multiple sclerosis because of the lack of muscular control.

Leber hereditary optic neuropathy is a condition related to changes in mitochondrial DNA. Although most DNA is packaged in chromosomes within the nucleus, mitochondria have a distinct mitochondrial genome composed of mtDNA. Mutations in the MT-ND1, MT-ND4, MT-ND4L, and MT-ND6 genes cause Leber hereditary optic neuropathy. These genes code for the NADH dehydrogenase protein involved in the normal mitochondrial function of oxidative phosphorylation. Oxidative phosphorylation uses a series of four large multi enzyme complexes, which are all embedded in the inner mitochondrial membrane to convert oxygen and simple sugars to energy. Mutations in any of the genes disrupt this process to cause a variety of syndromes depending on the type of mutation and other factors. It remains unclear how these genetic changes cause the death of cells in the optic nerve and lead to the specific features of Leber hereditary optic neuropathy. In Northern European populations about one in 9000 people carry one of the three primary LHON mutations. There is a prevalence of 1:30,000 to 1:50,000 in Europe. The LHON ND4 G1 1778 A mutation dominates as the primary mutation in most of the world with 70% of Northern European cases and 90% of Asian cases. Due to a Founder effect, the LHON ND6 T14484C mutation accounts for 86% of LHON cases in Quebec, Canada.

More than 50 percent of males with a mutation and more than 85 percent of females with a mutation never experience vision loss or related medical problems. The particular mutation type may predict likelihood of penetrance, severity of illness and probability of vision recovery in the affected. As a rule of thumb, a woman who harbors a homoplasmic primary LHON mutation has a −40% risk of having an affected son and a −10% risk of having an affected daughter.

Additional factors may determine whether a person develops the signs and symptoms of this disorder. Environmental factors such as smoking and alcohol use may be involved, although studies of these factors have produced conflicting results. Researchers are also investigating whether changes in additional genes, particularly genes on the X chromosome, contribute to the development of signs and symptoms. The degree of heteroplasmy, the percentage of mitochondria which have mutant alleles, may play a role. Patterns of mitochondrial alleles called haplogroup may also affect expression of mutations.

The eye pathology is limited to the retinal ganglion cell layer especially the maculopapillary bundle. Degeneration is evident from the retinal ganglion cell bodies to the axonal pathways leading to the lateral geniculate nucleii. Experimental evidence reveals impaired glutamate transport and increased reactive oxygen species (ROS) causing apoptosis of retinal ganglion cells. Also, experiments suggest that normal non LHON affected retinal ganglion cells produce less of the potent superoxide radical than other normal central nervous system neurons. Viral vector experiments which augment superoxide dismutase 2 in LHON cybrids or LHON animal models or use of exogenous glutathione in LHON cybrids have been shown to rescue LHON affected retinal ganglion cells from apoptotic death. These experiments may in part explain the death of LHON affected retinal ganglion cells in preference to other central nervous system neurons which also carry LHON affected mitochondria.

Without a known family history of LHON the diagnosis usually requires a neuro-ophthalmological evaluation and blood testing for mitochondrial DNA assessment. It is important to exclude other possible causes of vision loss and important associated syndromes such as heart electrical conduction system abnormalities. The prognosis for those affected left untreated is almost always that of continued significant visual loss in both eyes. Regular corrected visual acuity and perimetry checks are advised for follow up of affected individuals. There is beneficial treatment available for some cases of this disease especially for early onset disease. Also, experimental treatment protocols are in progress. Genetic counselling should be offered. Health and lifestyle choices should be reassessed particularly in light of toxic and nutritional theories of gene expression. Vision aides assistance and work rehabilitation should be used to assist in maintaining employment.

For those who are carriers of a LHON mutation, preclinical markers may be used to monitor progress. For example fundus photography can monitor nerve fiber layer swelling. Optical coherence tomography can be used for more detailed study of retinal nerve fiber layer thickness. Red green color vision testing may detect losses. Contrast sensitivity may be diminished. There could be an abnormal electroretinogram or visual evoked potentials. Neuron-specific enolase and axonal heavy chain neurofilament blood markers may predict conversion to affected status. Cyanocobalamin (a form of B12) should be avoided as it may lead to blindness in Leber's disease patients.

Avoiding optic nerve toxins is generally advised, especially tobacco and alcohol.

Certain prescription drugs are known to be a potential risk, so all drugs should be treated with suspicion and checked before use by those at risk. Ethambutol, in particular, has been implicated as triggering visual loss in carriers of LHON. In fact, toxic and nutritional optic neuropathies may have overlaps with LHON in symptoms, mitochondrial mechanisms of disease and management. Of note, when a patient carrying or suffering from LHON or toxic/nutritional optic neuropathy suffers a hypertensive crisis as a possible complication of the disease process, nitroprusside (trade name: Nipride) should not be used due to increased risk of optic nerve ischemia in response to this anti-hypertensive in particular.

Idebenonejias been shown in a small placebo controlled trial to have modest benefit in about half of patients. People most likely to respond best were those treated early in onset, a-Tocotrienol-quinone, a vitamin E metabolite, has had some success in small open label trials in reversing early onset vision loss. There are various treatment approaches which have had early trials or are proposed, none yet with convincing evidence of usefulness or safety for treatment or prevention including brimonidine, minocycline, curcumin, glutathione, near infrared light treatment, and viral vector techniques.

Idebenone is a short-chain benzoquinone that interacts with the mitochondrial electron transport chain to enhance cellular respiration. When used in individuals with LHON, it is believed to allow electrons to bypass the dysfunctional complex I. Successful treatment using idebenone was initially reported in a small number of patients.

Idebenone, combined with avoidance of smoke and limitation of alcohol intake, is the preferred standard treatment protocol for patients affected by LHON. Idebenone doses are prescribed to be taken spaced out throughout the day, rather than all at one time. For example, to achieve a dose of 900 mg per day, patients take 300 mg three times daily with meals. Idebenone is fat soluble, and may be taken with a moderate amount of dietary fat in each meal to promote absorption. It is recommended that patients on idebenone also take vitamin C 500 mg daily to keep idebenone in its reduced form, as it is most active in this state.

Currently, human clinical trials are underway at GenSight Biologies (ClinicalTrials.gov #NCT02064569) and the University of Miami (ClinicalTnals.gov # NCT02161380) to examine the safety and efficacy of mitochondrial gene therapy in LHON. In these trials, participants affected by LHON with the G11778A mutation will have a virus expressing the functional version of ND4—the gene mutated in this variant of LHON—injected into one eye. Preliminary results have demonstrated tolerability of the injections in a small number of subjects.

Stealth BioTherapeutics is presently investigating the potential use of bendavia (MTP-131), a mitochondrial protective agent, as a therapy for LHON. Bendavia helps stabilize cardiolipin—an important component of mitochondrial inner membranes—and has been shown to reduce damaging reactive oxygen species in animal models.

It is well established that mitochondrial dysfunction is a key factor in LHON neurodegenaeration (Hayashi & Cortopassi, 2015); ameliorating this dysfunction with A3AR agonist treatment will thus be a therapeutic approach for LHON.

F. Ototoxicity

A variety of drugs, including platinum complex cancer chemotherapeutics (e.g., cisplatin and oxaliplatin) and certain antibiotics (e.g., kanamycin and gentamicin) cause deafness, tinnitus, and hyperacusia) by damaging the inner hair cells and/or spiral ganglion neurons axon terminals. A drug-induced injury to the hair cell and neuronal mitochondria is a key factor (Guan, 2011; Devarajan et al, 2002); thus treatment to ameliorate the mitochondrial insult with A3AR agonist treatment will thus be a treatment for drug-induced otototoxicity.

### II. A3 ADENOSINE RECEPTORS

The A₃ adenosine receptor (A3AR) belongs to the Gi-protein-associated cell membrane receptors. Activation of these receptors inhibits adenylate cyclase activity, inhibiting cAMP formation, leading to the inhibition of PKA expression and initiation of a number of downstream signaling pathways. A variety of agonists to this receptor subtype have been synthesized including IB-MECA (N⁶-(3-iodobenzyl)-adenosine-5′-N-methyluronamide) and its chlorinated form Cl-IB-MECA (2-chloro-N⁶-(3-iodobenzyl)-adenosine-5′-N-methyluronamide). Methanocarba adenosine derivatives, including but not limited to MRS5698, MRS5980, MRS 7144 and MRS7154 are among the most potent and specific presently known A₃AR agonists.

The present inventor has previously described the use of A₃AR agonists as pharmaceutical compounds in treatments against pain (U.S. Patent Publication 2012/0270829). In particular, A₃AR agonists have been found to be effective in the treatment of neuropathic pain, especially with regard to blocking and/or reversing the development of chemotherapy-induced neuropathic pain (CIPN) and nerve-injury-derived neuropathic pain. Thus, A₃AR agonists were proposed for use in shielding cancer patients from the pain due to chemotherapeutic agents and other causes. Moreover, A₃AR agonists and market-leading analgesics have been found to exhibit a synergistic effect in the treatment of neuropathic pain. However, A₃AR agonists have no effect on normal pain behavior {i.e., unlike opioids which block acute nociception in response to severe noxious stimuli, for example using a tail flick assay, A₃AR agonists have no effect). In addition when given acutely together, an A₃AR agonist will not potentiate the antinociceptive effect of an opioid in models of acute nociception.

### III. A3AR AGONISTS

It can be confirmed that a compound has an A₃AR activity by known methods. Examples of A₃AR agonists that may be used in accordance with the present include, but are not limited to, N⁶-benzyladenosine-5′-N-methyluronamides such as N⁶-(3-iodobenzyl)-adenosine-5′-N-methyluronamide, also known as IB-MECA, and 2-Chloro-N⁶-(3-iodobenzyl)-adenosine-5′-N-methyluronamide (also known as 2-Cl-IB-MECA; (N)-methanocarba nucleosides such as (1R,2R,3S,4R)-4-(2-chloro-6-((3-chlorobenzyl)amino)-9H-purin-9-yl)-2,3-di-hydroxy-N-methylbicyclo[3.1.0]hexane-1-carboxamide (also known as CF502, Can-Fite Biopharma, MA); (2S,3S,4R,5R)-3-amino-5-[6-(2,5-dichlorobenzylamino)purin-9-yl]-4-hydroxy-tetrahydrofuran-2-carboxylic acid methylamide (also known as CP-532,903); (1′S,2′R,3′S,4′R,5′S)-4-(2-chloro-6-(3-chlorobenzylamino)-9H-purin-9-yl)˜2,3-dihydroxy-N-methylbicyclo[3.1.0]hexane-1-carboxatnide (also known as MRS-3558); (1′R,2′R,3′S,4′R,5′S)-4-{2-chloro-6-[(3-iodophenylmethyl)amino]purin-9-yl-}-1-(methylaminocarbonyl)-bicyclo[3.1.0]hexane-2,3-diol (also known as MRS1898); and 2-Dialkynyl derivatives of (N)-methanocarba nucleosides, 2-(arylethynyl)adenine and N(6)-methyl or N(6)-(3-substituted-benzyl), N(6)-methyl 2-(halophenylethynyl) analogues, polyaromatic 2-ethynyl N(6)-3-chlorobenzyl analogues, such as 2-p-biphenylethynyl MRS5679 and fluorescent 1-pyrene adduct MRS5704, as well as MRS5678.

A particular embodiment of this disclosure involves the use of a highly-selective A₃AR agonist, including but not limited to an adenosine methanocarba derivative described in Tosh et al. (2014), including but not limited to MRS56908, MRS5980, MRS7144, and MRS7154 given together with paclitaxel or oxaliplatin anti-cancer chemotherapy in order to prevent CIPN.

Also included are A₃AR allosteric modulators which enhance the receptor activity in the presence of the native ligand, such as 2-cyclohexyl-N-(3,4-dichlorophenyl)-1H-imidazo[4,5-c]quinolin-4-amine (also known as CF602, Can-Fite). However, the above-listed A₃AR agonists are by no means exclusive and other such agonists may also be used. The administration of A₃AR agonists covalently bound to polymers is also contemplated. For example, A₃AR agonists may be administered in the form of conjugates where an agonist is bound to a polyamidoamine (PAMAM) dendrimer. The following table illustrates additional A₃AR agonists that can be employed in accordance with the present disclosure:

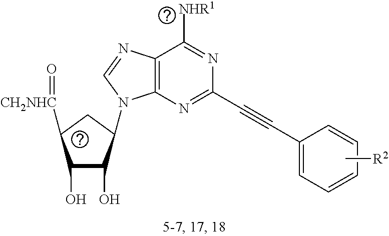

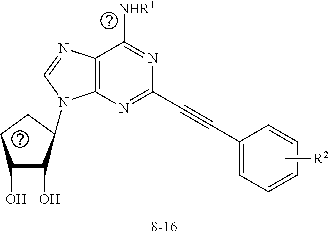

Affinity (K_{D} nm) or % inhibition (italic)^{a,b}

| Cmpd | R¹ | R² | Species | A₁ | A_{2A} | A₃ | % efficacy |
|---|---|---|---|---|---|---|---|
| 5^{d} | 3-Cl•Bn | H | h | (20% ± 3%) | (27% ± 3%) | 1.34 ± 0.30 | 101 ± 5.9 |
| | | | m | (50% ± 5% | (2% ± 1%) | 1.23 ± 0.14 | ND |
| 6 | 3-Cl•Bn | 4-SO₃H | h | 383 ± 7.5 | (23% ± 3%) | 11.1 ± 1.6 | 98.6 ± 5.7 |
| | | | m | 35.1 ± 5.5 | (14% ± 4%) | 9.68 ± 0.15 | 95.7 ± 19.1 |
| 7 | 3-Cl•Bn | 3-SO₃H | h | (16% ± 3%) | (7% ± 6%) | 1.90 ± 0.03 | 98.2 ± 6.7 |
| | | | m | (15% ± 2%) | (1% ± 1%) | 11.3 ± 1.9 | 89.3 ± 7.1 |
| 8^{d} | Me | H | h | (18% ± 1%) | (18% ± 3%) | 5.48 ± 1.23 | 12.6 ± 4.0 |
| | | | m | 3800 ± 780 | (8% ± 3%) | 1530 ± 240 | ND |
| 9^{d} | Et | H | h | (36% ± 4%) | (42% ± 4%) | 5.02 ± 2.19 | 0.8 ± 5.2 |
| | | | m | (49% ± 6%) | (49% ± 2%) | 1480 ± 170 | ND |
| 10^{d} | Et | 2-Cl | h | (25% ± 11%) | (17% ± 6%) | 5.8 ± 2.08 | 7.0 ± 5.2 |
| | | | m | (47% ± 8%) | (11% ± 1%) | (50% ± 9%) | ND |
| 11^{d} | 3-Cl•Bn | H | h | (37% ± 4%) | 680 ± 170 | 39.0 ± 20.0 | 13.8 ± 5.1 |
| 12^{d} | 3-Cl•Bn | 3-Cl | h | (26% ± 3%) | 1800 ± 310 | 210 ± 40 | 4.5 ± 4.9 |
| 13^{d} | 3-Cl•Bn | 4-Ph | h | (48% ± 4%) | (12% ± 7%) | 54.0 ± 7.0 | 3.5 ± 3.2 |
| | | | m | 1110 ± 220 | (0%) | 255 ± 77 | ND |
| 14^{d} | Ph(CH₂)₂ | H | h | (30% ± 8%) | (22% ± 5%) | 20.0 ± 6.0 | 4.1 ± 1.2 |
| | | | m | (39% ± 6%) | (13% ± 2%) | 480 ± 90 | 14.3 ± 6.1 |
| 15^{d} | Ph₂CHCH₂ | 2-Cl | h | (26% ± 4%) | (22% ± 2%) | 140 ± 30 | 2.6 ± 1.3 |
| | | | m | (23% ± 1%) | (16% ± %) | (54 ± 3%) | ND |
| 16 | 4-SO₃H•Ph(CH₂)₂ | H | h | (10% ± 5%) | (15% ± 3%) | 30.2 ± 4.3 | 7.2 |
| | | | m | (18% ± 2%) | (1% ± 2%) | 3920 ± 1190 | ND |
| 17 | Ph(CH₂)₂ | H | h | (11% ± 3%) | (32% ± 4%) | 1.23 ± 0.57 | 105.3 ± 9.8 |
| | | | m | (25% ± 5%) | (1% ± 1%) | 8.75 ± 2.12 | 114.6 ± 14.6 |
| 18 | 4-SO₃H•Ph(CH₂)₂ | H | h | (9% ± 5%) | (1% ± 1%) | 12.1 ± 1.0 | 93.8 ± 7.1 |
| | | | m | (7% ± 2%) | (0%) | 71.1 ± 13.0 | ND |

^{a}Binding in membranes prepared from CHO or HEK293 (A_{2A} only) cells stably expressing one of three
hAR subtypes. The binding affinity for A₁AR₁ and A₃AR was expressed as Kᵢ values (η - 3-4) using
agonist radioligands [³H]N⁶-R-phenylisopropyladenosine 40. [³H]2-[p(2-carboxyethyl)phenylethylamino]-5′-
N-ethylcarboxamidoadenosine 41, or [¹²⁵I]N⁶-(4-amino-3-iodobenzyl)adenosine-5′-N-methyluronamide 42,
respectively. A percent in parentheses refers to inhibition of bind 10 μM.
^{b}Binding in membranes preared from HEK293 cells stably expressing one of three mAR subtypes. Radioligand
used were [¹²⁵I]N⁶-(4-amino-3-iodobenzyl)adenosine-5¹-N-methyloronamide 43 (A₁AR and A₃AR) and [³H]2-
[p-(2-carboxyethyl)phenylethylamino]-5′-N-ethylcarboxamidoadenosine 41 (A_{2A}AR). The data (η - 3-4) are express
as Kᵢ values. A percent in parentheses refers to inhibition of binding 10 μM.
^{c}Efficacy, expressed as a percentage of the maximal effect of either 5′-N-ethylcarboxamidoadenosine 43 (hA₃AR₅)
or N⁶-(3-iodobenzyl)adenosine-5′-N-methyloronamide 1a (mA₃AR₅) to inhibit forskolin-stimulated cAMP
production, was determined in cAMP assays us hA₃AR-transfected CHO cells or mA₁AR-transfected HEK cells.
In studies with the hA₃AR, maximal efficacies of 43 and the test compounds were estimated by measuring the
extent of inhibition of forskolin-stimulated cAMP accumulation produced each at a concentration of 10 μM. In
studies with the mA₃AR, maximal efficacies of 1a and test compounds were determined from concentration-effect curves.
Data are expressed as mean ± SEM (η - 3-5).
ND: not deteremined.
^{d}Compounds 5 and 8-15 were prepared and tested for binding at the hAR₅ in ref 26.

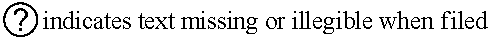

### IV. OTHER DISEASE STATES

A. Spinocerebellar Degeneration

Spinocerebellar ataxia (SCA) or also known as Spinocerebellar atrophy or Spinocerebellar degeneration, is a progressive, degenerative, genetic disease with multiple types, each of which could be considered a disease in its own right. An estimated 150,000 people in the United States are diagnosed with Spinocerebellar Ataxia. SCAs are the largest group of this hereditary, progressive, degenerative and often fatal neurodegenerative disorder. There is no known effective treatment or cure. Spinocerebellar Ataxia can affect anyone of any age. The disease is caused by either a recessive or dominant gene. In many cases people are not aware that they carry the ataxia gene until they have children who begin to show signs of having the disorder.

Spinocerebellar ataxia (SCA) is one of a group of genetic disorders characterized by slowly progressive incoordination of gait and is often associated with poor coordination of hands, speech, and eye movements. A review of different clinical features among SCA subtypes was recently published describing the frequency of non-cerebellar features, like parkinsonism, chorea, pyramidalism, cognitive impairment, peripheral neuropathy, seizures, among others. As with other forms of ataxia, SCA frequently results in atrophy of the cerebellum, loss of fine coordination of muscle movements leading to unsteady and clumsy motion, and other symptoms.

The symptoms of an ataxia vary with the specific type and with the individual patient. In general, a person with ataxia retains full mental capacity but progressively loses physical control.

There is no cure for spinocerebellar ataxia, which is considered to be a progressive and irreversible disease, although not all types cause equally severe disability. In general, treatments are directed towards alleviating symptoms, not the disease itself. Many patients with hereditary or idiopathic forms of ataxia have other symptoms in addition to ataxia. Medications or other therapies might be appropriate for some of these symptoms, which could include tremor, stiffness, depression, spasticity, and sleep disorders, among others. Both onset of initial symptoms and duration of disease are variable. If the disease is caused by a polyglutamine trinucleotide repeat CAG expansion, a longer expansion may lead to an earlier onset and a more radical progression of clinical symptoms. Typically, a person afflicted with this disease will eventually be unable to perform daily tasks (ADLs). However, rehabilitation therapists can help patients to maximize their ability of self-care and delay deterioration to certain extent. Stem cell research has been sought for a future treatment.

It is well established that mitochondrial dysfunction is a key factor in SCA neurodegenaeration (Matilla-Duenas et al, 2014); ameliorating this dysfunction with A3AR agonist treatment is thus a potential therapeutic approach for SCA.

B. Diabetic Neuropathy

Diabetic neuropathies are nerve damage disorders associated with diabetes mellitus. Relatively common conditions which may be associated with diabetic neuropathy include third nerve palsy; mononeuropathy; mononeuropathy multiplex; diabetic amyotrophy; a distal symmetrical dying-back sensorimotor polyneuropathy with or without a neuropathic pain component; autonomic neuropathy; and thoracoabdominal neuropathy.

Diabetic neuropathy affects all peripheral nerves including pain fibers, motor neurons and the autonomic nervous system. It therefore can affect all organs and systems, as all are innervated.

There are several distinct syndromes based upon the organ systems and members affected, but these are by no means exclusive. A patient can have sensorimotor and autonomic neuropathy or any other combination. Signs and symptoms vary depending on the nerve(s) affected and may include symptoms other than those listed. Symptoms usually develop gradually over years.

Symptoms may include the following: trouble with balance, numbness and tingling of extremities, dysesthesia, diarrhea, erectile dysfunction, urinary incontinence, facial, mouth and eyelid drooping, vision changes, dizziness, muscle weakness, difficulty swallowing, speech impairment, fasciculation, anorgasmia, retrograde ejaculation and burning or electric pain Diabetic peripheral neuropathy is the most likely diagnosis for someone with diabetes who has pain in a leg or foot, although it may also be caused by vitamin B₁₂ deficiency or osteoarthritis. A recent review in the Journal of the American Medical Association's “Rational Clinical Examination Series” evaluated the usefulness of the clinical examination in diagnosing diabetic peripheral neuropathy. While the physician typically assesses the appearance of the feet, presence of ulceration, and ankle reflexes, the most useful physical examination findings for large fiber neuropathy are an abnormally decreased vibration perception to a 128-Hz tuning fork (likelihood ratio (LR) range, 16-35) or pressure sensation with a 5.07 Semmes-Weinstein monofilament (LR range, 11-16). Normal results on vibration testing (LR range, 0.33-0.51) or monofilament (LR range, 0.09-0.54) make large fiber peripheral neuropathy from diabetes less likely. Combinations of signs do not perform better than these 2 individual findings. Nerve conduction tests may show reduced functioning of the peripheral nerves, but seldom correlate with the severity of diabetic peripheral neuropathy and are not appropriate as routine tests for the condition.

With the exception of tight glucose control, treatments are for reducing pain and other symptoms. Options for pain control include tricyclic antidepressants (TCAs), serotonin-norepinephrine reuptake inhibitors (SNRIs), antiepileptic drugs (AEDs) and capsaicin cream. A systematic review concluded that “tricyclic antidepressants and traditional anticonvulsants are better for short term pain relief than newer generation anticonvulsants.” A further analysis of previous studies showed that the agents carbamazepine, venlafaxine, duloxetine and amitriptyline were more effective than placebo, but that comparative effectiveness between each agent is unclear.

The only three drugs approved by the FDA for diabetic peripheral neuropathy are the antidepressant duloxetine, the anticonvulsant pregabalin, and the long-acting opioid tapentadol ER. Before trying a systemic medication, some doctors recommend treating localized diabetic peripheral neuropathy with lidocaine patches.

It is well established that mitochondrial dysfunction is a key factor in the neurodegenaeration seen with diabetic peripheral neuropathy (Zenker et al, 2013); ameliorating this dysfunction with A3AR agonist treatment is thus a potential therapeutic approach for diabetic neuropathy.

### V. PHARMACEUTICAL FORMULATIONS AND ROUTES OF ADMINISTRATION

Where clinical applications in treating pain are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render materials stable and allow for interaction with target cells. Aqueous compositions of the present disclosure comprise an effective amount of the agent, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the compositions of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, transdermal, intradermal, subcutaneous, intramuscular, intraperitoneal, intrathecal or intravenous injection. Such compositions would normally be administered as pharmaceutically acceptable compositions, described supra. Of particular interest are routes suitable for blood-brain barrier transport.

With regard to transdermal delivery, a patch is particularly contemplated. There are five main types of transdermal patches. In the Single-layer Drug-in-Adhesive, the adhesive layer of this system also contains the drug. In this type of patch the adhesive layer not only serves to adhere the various layers together, along with the entire system to the skin, but is also responsible for the releasing of the drug. The adhesive layer is surrounded by a temporary liner and a backing. In Multi-layer Drug-in-Adhesive, the multi-layer drug-in adhesive patch is similar to the single-layer system in that both adhesive layers are also responsible for the releasing of the drug. One of the layers is for immediate release of the drug and other layer is for control release of drug from the reservoir. The multi-layer system is different however that it adds another layer of drug-in-adhesive, usually separated by a membrane (but not in all cases). This patch also has a temporary liner-layer and a permanent backing.

Unlike the Single-layer and Multi-layer Drug-in-adhesive systems, the reservoir transdermal system has a separate drug layer. The drug layer is a liquid compartment containing a drug solution or suspension separated by the adhesive layer. This patch is also backed by the backing layer. In this type of system the rate of release is zero order.

The Matrix system has a drug layer of a semisolid matrix containing a drug solution or suspension. The adhesive layer in this patch surrounds the drug layer partially overlaying it. Also known as a monolithic device.

In Vapor Patches, the adhesive layer not only serves to adhere the various layers together but also to release vapour. The vapour patches are new on the market and they release essential oils for up to 6 hours. The vapour patches release essential oils and are used in cases of decongestion mainly. Other vapour patches on the market are controller vapour patches that improve the quality of sleep. Vapour patches that reduce the quantity of cigarettes that one smokes in a month are also available on the market.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present disclosure may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The methods of the disclosure can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, and mice.

### VI. COMBINATION THERAPIES

Treating CIPN may involve the co-administration of an A₃AR agonist and a chemotherapeutic. The agents would be provided in a combined amount effective to treat the cancer or viral disease while reducing neuropathy. This process may involve contacting the patient with the agents at the same time. This may be achieved by contacting the patient with a single composition or pharmacological formulation that includes both agents, or by contacting the cell with two distinct compositions or formulations, at the same time, wherein one composition includes the A₃AR agonist and the other includes the chemotherapeutic.

Alternatively, the A₃AR treatment may precede or follow the chemotherapeutic by intervals ranging from minutes to weeks. In embodiments where the chemotherapeutic and the A3AR agonist are applied separately to the subject, one would generally ensure that a significant period of time did not expire between each delivery, such that the therapies would still be able to exert an advantageously combined effect on the subject. In such instances, it is contemplated that one would administer both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the A₃AR agonist or the chemotherapeutic therapy will be desired. Various combinations may be employed, where the A₃AR agonist is “A,” and the other agent is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations, including chronic and continuous dosing of one or both agents, are contemplated.

### VII. EXAMPLES

The following examples are included to demonstrate particular embodiments of the disclosure. It should be appreciated by those of skill in the art that the techniques disclosed in the examples which follow represent techniques discovered by the inventor to function well in the practice of the disclosure, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Chemotherapeutic-induced peripheral neuropathy. Oxaliplatin (Oncology Supply; Dothan, Ala.) or its vehicle (5% dextrose) was injected i.p. on 5 consecutive days (DO-4) for a final cumulative dose of 10 mg/kg.³ MRS 5698 or its vehicle (0.01% dimethylsulfoxide in phosphate-buffered saline, pH 7.4) were administered i.p. (0.1 mg/kg/d) concomitantly with oxaliplatin. Behavior was measured for 25 days following the first dowse of oxaliplatin using manual vonFrey filaments & the “up-down” method.

Raman microscopy. CHO & BV2 cells were grown on quartz slides in Fluorobrite DMEM (Life Technologies) & treated for 1 h with MRS5698 (200-250 nM) or MRS5980 (1 μM). Individual cells were located by brightfield imaging using a 63× (NA=1.0) dipping objective on a WITec alpha300R microscope.

Western blot. Tissues were homogenized in a sucrose buffer (0.25 M sucrose, 1 mM EDTA, 20 mM Hepes-NaOH, pH 7.4) & differentially centrifuged (800×g, 10000×g, & 8000×g) to generate a crude mitochondrial pellet. Crude mitochondrial pellets from spinal cord, sciatic nerve, liver & PBL were further purified on 15, 20, 25, 30, 44% Optiprep gradient at 100000×g. A₃AR (Bioss), calreticulin (Cell Signaling) & VDAC1 (Cell Signaling) in mitochondrial pellets were detected by chemiluminescence.

Stimulated Emission Depletion Microscopy (STED) CTXTNA2 & BV2 cells were grown on glass coverslips, then fixed with 4% paraformaldhyde & labeled for A₃AR (rabbit, 1:200, Bioss) & TOMM20 (mouse, 1:200, Abcam). Images were collected on a Leica TCS SP8 STED 3× instrument (Leica Microsystems, Exton, Pa., USA) using 592 nm & 775 nm depletion lasers for Oregon Green 488 & Cy3 channels respectively. Images were post-processed by deconvolution with SVI Huygens Professional software (SVI, Netherlands) & either single plane images or 3D surface rendering was performed using Leica LASX software.

Transmission Electron Microscopy. Purified spinal mitochondria or saphenous nerve tissue were fixed in 4% paraformaldehyde & 0.1M cacodylate, dehydrated in cold ethanol series & embedded in LR White. Cross-sections (90 nm) were mounted on Formvar-coated nickel slots grids, treated (10 min) with 0.1M sodium citrate, washed & incubated in 3% sodium metaperiodate (10 min). Sections were blocked & labeled for A₃AR (rabbit, 1:100, Bioss) or COXIV (mouse, 1:50, Abcam) & 25 nm anti-rabbit or 15 nm anti-mouse colloidal gold.

Sections were examined using Hitachi H-7500 transmission electron microscope. Digitized images were obtained & archived by an ORCA camera with IC-PCI framegrabber & AMT 12-HR software.

Mitochondrial membrane potential (ΔΨm). Mitochondria from mouse & rat liver were isolated for flow cytometry as previously described. Mitochondria (57 μg) were loaded with Mitotracker DeepRed FM (50 nM) & TMRM (100 nM). Mitochondria were then treated for 5 min with MRS5980 (10 μM) or vehicle then stimulated with ADP (1 mM) or Ca²⁺ (0-15 μM) for 1 min. Mitochondria (10,000 counts) were detected using Mitotracker Deep Red FM (Abs/EM 644/665 nm) signal & the ΔΨm state determined by the dynamic median fluorescence signal of TMRM using a FACSCanto II (BD Biosciences).

ATP assays. Saphenous nerves were harvested from rats 25 days after the initiation of chemotherapy treatments, then minced & teased apart as previously described. Saphenous nerve explants were transferred to MiR05 respiratory buffer & baseline ATP samples were taken after 5 min or after 15 min treatment with MRS5980 or vehicle. ADP (1 mM), glutamate (5 mM), succinate (5 mM), maleate (5 mM) were added for 5 min & samples were taken for ATP. ATP levels were measured by a flash luciferin-luciferase assay (Promega Enliten ATP Assay; Promega, Madison, Wis.) & normalized to citrate synthase activity (Sigma, St Louis, Mo.) in explants homogenates.

### Example 2—Results and Discussion

Data are shown below in Table 1, in the figures and legends, and in Appendix A. These data show that A₃AR is a novel mitochondrial G protein-coupled receptor. The function of mitochondrial A₃AR signaling may include sustaining mitochondrial bioenergetics. Thus, the mitoprotective effects of A₃AR agonists observed in the context of peripheral neuropathy may result from direct A₃AR signaling in the mitochondria. Mitochondrial A₃AR may prove to be a novel therapeutic target for the treatment of peripheral neuropathies & mitochondrial disorders that lead to neurodegeneration or degeneration of the cochlear hair cells.

TABLE 1
Bioinformatic analysis reveals potential mitochondrial targeting.

| | | | # Of | | | PROB. Of export |
|---|---|---|---|---|---|---|
| Protein | Species | Accession# | amino acids | Net charge | Cleavage site | to mitochondria |
| A₃AR | mouse | Q61618.2 | 319 | +10 | Not predictable | 0.0136 |
| | rat | P28647.3 | 320 | +13 | 173 | 0.0501 |
| | human | P0DMS8.1 | 318 | +10 | 161 | 0.0950 |

Established mitochondrial proteins

| | | | | | | |
|---|---|---|---|---|---|---|
| | mouse | NP_035824.1 | 283 | +3 | not predictable | 0.2840 |
| VDAC1 | rat | NP_112643.1 | 283 | +3 | not predictable | 0.2840 |
| | human | NP_003365.1 | 283 | +3 | not predictable | 0.3892 |
| | mouse | NP_077176.1 | 145 | +3 | not predictable | 0.0644 |
| TOMM20 | rat | NP_690918.1 | 145 | +3 | not predictable | 0.0686 |
| | human | NP_055580.1 | 145 | +3 | not predictable | 0.0660 |
| | mouse | NP_034071.2 | 206 | +12 | 61 | 0.9840 |
| COXIV | rat | NP_058898.1 | 169 | +7 | 24 | 0.9690 |
| | human | AAA52059.1 | 169 | +8 | 23 | 0.9807 |

Mitochondrial G protein-coupled receptors

| | | | | | | |
|---|---|---|---|---|---|---|
| | mouse | NP_031752.1 | 473 | +6 | not predictable | 0.4064 |
| Cannabinoid | rat | NP_036916.1 | 473 | +6 | not predictable | 0.4064 |
| Receptor 1 | | | | | | |
| | human | P21554.1 | 472 | +5 | not predictable | 0.3934 |
| | mouse | AAH79624.1 | 326 | +6 | 43 | 0.1143 |
| A₁AR | rat | NP_058851.2 | 326 | +7 | 43 | 0.1137 |
| | human | NP_001041695.1 | 326 | +7 | 43 | 0.1369 |

Established non-mitochondrial proteins

| | | | | | | |
|---|---|---|---|---|---|---|
| Calreticulin | mouse | AAH03453.1 | 416 | −55 | not predictable | 0.0028 |
| (ER, soluble) | | | | | | |
| | rat | CAA55890.1 | 416 | −55 | not predictable | 0.0022 |
| | human | NP_004334.1 | 417 | −59 | not predictable | 0.0017 |
| | mouse | NP_001103970.1 | 591 | −63 | not predictable | 0.0010 |
| Calnexin | rat | NP_742005.1 | 591 | −59 | not predictable | 0.0030 |
| (ER, membrane) | | | | | | |
| | human | NP_001019820.1 | 592 | −62 | not predictable | 0.0014 |

Analyzed using MitoProtII v.1.101 (M. G. Claros, P. Vincens. Computational method to predict mitochondrially imported proteins & their targeting sequences. (1996) Eur. J. Biochem. 241, 779-786

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to 0 the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to 5 those skilled in the art are deemed to be within the spirit, scope and concept of the disclosure as defined by the appended claims.

### VIII. REFERENCES

The following references, to the extent that they provide exemplary procedural or other details supplementary to those set forth herein, are specifically incorporated herein by reference:
Bennett et al, “Mitotoxicity in distal symmetrical sensory peripheral neuropathies”. Nature Review Neurology 10(6):326-36, 2014. doi: 10.1038/nrneurol.2014.77.
Carvalho et al, “The role of mitochondrial disturbances in Alzheimer, Parkinson and Huntington diseases. Expert Review in Neurotherapy 15(8):867-84; 2015.
Cozzolino et al, “Mitochondrial dynamism and the pathogenesis of Amyotrophic Lateral Sclerosis”. Fronteirs in Cellular Neuroscience 10; 9:31, 2015. doi: 10.3389/fncel.2015.00031.
D'Amour, Journal of Pharmacology and Experimental Therapeutics 72:74-79, 1941. Devarajan et al, “Cisplatin-induced apoptosis in auditory cells: role of death receptor and mitochondrial pathways”. Hearing Research 174(1-2):45-54, 2002.
Guan, “Mitochondrial 12S rRNA mutations associated with aminoglycoside ototoxicity” Mitochondrion 11 (2): 237-45, 2011.
Hayashi & Cortopassi. “Oxidative stress in inherited mitochondrial diseases”. Free Radical Biology and Medicine 88(Pt A): 10-7, 2015. doi:0.1016/j.freeradbiomed.2015.05.039.
Foley, Anticancer Drugs 6:Suppl 3:4-13, 1995.
Fredholm et al., Pharmacological Reviews 53:527-552, 2001.
Fredholm et al., Pharmacological Reviews 63: 1-34, 2011.
King et al, Pain 132: 154-168, 2007.
Liu et al, Brain, Behavior, and Immunity 25: 1223-1232, 2011. Matilla-Duenas A et al, Consensus paper: pathological mechanisms underlying neurodegeneration in spinocerebellar ataxias. Cerebellum 13(2):269-302, 2014. doi: 10.1007/s12311-013-0539-y.
Muscoli et al., The Journal of Neuroscience 30: 15400-15408, 2010.
Remington's Pharmaceutical Sciences, 15th Edition.
Renfrey et al., Nature Review Drug Discovery 2: 175-176, 2003. Tosh et al, “In vivo phenolypic screening for treating chronic neuropathic pain: modification of C2-arylethynyl group of conformationally constrained A3 adenosine receptor agonists,” Journal of Medicinal Chemistry 57:9901-9914, 2014.
Vera-Portocarrero et al., Pain 129:35-45, 2007. Zenker et al, “Novel pathogenic pathways in diabetic neuropathy”. Trends in Neuroscience 36(8):439-49, 2013. doi: 10.1016/j.tins.2013.04.008.

1. A method of treating chemotherapy-induced peripheral neuropathy (CIPN) in a subject comprising administering to said subject an A₃AR agonist.

2. The method of claim 1, wherein said CIPN is due to an anti-cancer chemotherapy.

3. The method of claim 2, wherein said anti-cancer chemotherapy is selected from the group consisting of a taxane chemotherapeutic, a platinum-complex chemotherapeutic, a vinca alkaloid chemotherapeutic, and a proteasome inhibitor chemotherapeutic.

4. The method of claim 1, wherein said CIPN is due to an anti-viral chemotherapy.

5. (canceled)

6. A method of treating diabetic peripheral neuropathy in a subject comprising administering to said subject an A₃AR agonist.

7. A method of treating a neurodegeneration in a subject comprising administering to said subject an A₃AR agonist.

8. The method of claim 7, wherein neurodegeneration is due to Alzheimer's disease, Parkinson's disease, Huntington's disease, amyotrophic lateral sclerosis, or Leber's optic neuropathy.

9. A method of treating drug-induced ototoxicity in a subject comprising administering to said subject an A₃AR agonist.

10. The method of claim 9, wherein the drug-induced ototoxicity is deafness, tinnitus, or hyperacusia.

11. A method of treating spinocerebellar degeneration in a subject comprising administering to said subject an A₃AR agonist.

12. The method of claim 1, wherein said A₃AR agonist is selected from the group consisting of IB-MECA, Cl-IB-MECA, and an adenosine methanocarba derivative.

13.-25. (canceled)

26. The method of claim 1, further comprising administering to said subject an additional therapy that treats CIPN.

27. The method of claim 12, wherein said A₃AR agonist is selected from the group consisting of MRS5698, MRS5980, MRS7144 and MRS7154.

28. The method of claim 6, wherein said A₃AR agonist is selected from the group consisting of IB-MECA, Cl-IB-MECA, and an adenosine methanocarba derivative.

29. The method of claim 28, wherein said A₃AR agonist is selected from the group consisting of MRS5698, MRS5980, MRS7144 and MRS7154.

30. The method of claim 7, wherein said A₃AR agonist is selected from the group consisting of IB-MECA, Cl-IB-MECA, and an adenosine methanocarba derivative.

31. The method of claim 30, wherein said A₃AR agonist is selected from the group consisting of MRS5698, MRS5980, MRS7144 and MRS7154.

32. The method of claim 9, wherein said A₃AR agonist is selected from the group consisting of IB-MECA, Cl-IB-MECA, and an adenosine methanocarba derivative.

33. The method of claim 32, wherein said A₃AR agonist is selected from the group consisting of MRS5698, MRS5980, MRS7144 and MRS7154.

34. The method of claim 11, wherein said A₃AR agonist is selected from the group consisting of IB-MECA, Cl-IB-MECA, and an adenosine methanocarba derivative.

35. The method of claim 34, wherein said A₃AR agonist is selected from the group consisting of MRS5698, MRS5980, MRS7144 and MRS7154.
